SEQUENCE LISTING (41 sequences)

SEQ ID NO: 1 - protein, 187 aa, Unknown
  RING Domain
RKRRGGAINSRQAQKRTREATSTPEISLEAEPIELVETAGDEIVDLTCESLEPVVVDLTHNDSVVIVDERRRPRRNARRLPQDHADSCVVSSDDEELSRDRDVYVTTHTPRNARDEGATGLRPSGTVSCPICMDGYSEIVQNGRLIVSTECGHVFCSQCLRDSLKNANTCPTCRKKINHKRYHPIYI

SEQ ID NO: 2 - protein, 66 aa, Unknown
  RING Domain
RPSGTVSCPICMDGYSEIVQNGRLIVSTECGHVFCSQCLRDSLKNANTCPTCRKKINHKRYHPIYI

SEQ ID NO: 3 - protein, 187 aa, Unknown
  RING Domain
RKRRGGTVNSRQTQKRTRETTSTPEVSLETEPIELVETVGDEIVDLTCESLEPVVVDLTHNDSVVIVEERRRPRRNGRRLRQDHADSCVVSSDGEELSRDKDVYVTTHTPRSTKDDGATGPRPSGTVSCPICMDGYSEIVQNGRLIVSTECGHVFCSQCLRDSLKNANTCPTCRKKINHKRYHPIYI

SEQ ID NO: 4 - protein, 66 aa, Unknown; identical to SEQ ID NO: 2
  RING Domain

SEQ ID NO: 5 - protein, 189 aa, Unknown
  RING Domain
RKRRGGSANSRQAQKRSRLIASTTEMASEGEPIELEESAGEEVVDLTCESSDPVVVDLTHNDSIVIVEENQQRRRNLRLRGQRQSDSCVLSSDDEDETRDNDVYVTDKVSRELGPLEDETASSKPSGTVSCPICMDGYSEIVQSGRLIVSTKCGHVFCSQCLRGSLRNANSCPTCRKKLTHRQYHPIYI

SEQ ID NO: 6 - protein, 66 aa, Unknown
  RING Domain
KPSGTVSCPICMDGYSEIVQSGRLIVSTKCGHVFCSQCLRGSLRNANSCPTCRKKLTHRQYHPIYI

SEQ ID NO: 7 - protein, 186 aa, Unknown
  RING Domain
RKRRSGTTNSRQAQKRERLVGPTSDMTSETEPIELVESAGEEVVDLTCESTEPVVVDLTHNDPVVFIEENRRQRRNPRISRGQPDSCVLSSDDEEIRDNDVYVTTENPSRESPDPAGFGEKPSGTVSCPICMDGYSEILQSGRLIVSTKCGHVFCSQCLRDALRNASSCPTCRKKLNQKQYHPIYI

SEQ ID NO: 8 - protein, 65 aa, Unknown
  RING Domain
PSGTVSCPICMDGYSEILQSGRLIVSTKCGHVFCSQCLRDALRNASSCPTCRKKLNQKQYHPIYI

SEQ ID NO: 9 - protein, 181 aa, Unknown
  RING Domain
GSMVNSRQAREFHTAGERGPTPQTVLEAELIELGESDEVVDLTCESLEPAVIDLTHQDCVVITEERRRPRGNTRSLQGQTGSCVVSSDKELMRDRDVYVTHSAYHNALEEETLSCTLPGYIQCRICMDGYSEIELSRRHIYSTDCGHIFCSQCLCTSLKYTKTCPVCLKKIGCRQYHRIYL

SEQ ID NO: 10 - protein, 65 aa, Unknown
  RING Domain
LPGYIQCRICMDGYSEIELSRRHIYSTDCGHIFCSQCLCTSLKYTKTCPVCLKKIGCRQYHRIYL

SEQ ID NO: 11 - protein, 183 aa, Unknown
  RING Domain
VTQRKRRTSTTCSRRGNSKRNRAQMSQTVMETIDVLENDRTNSEDVVDLTCEGSEPAVVDLTNNDSIVVVEDGVQRRVGPCTESYVLSSDEEEESSLRLVYVTSPGLLSSLRDSSRARSTGAISCPVCMDVYSEIMDSGRLMVSTKCGHLFCSQCIRDSLSRAHSCPTCRKKLTHKQYHPIYI

SEQ ID NO: 12 - protein, 66 aa, Unknown
  RING Domain
RSTGAISCPVCMDVYSEIMDSGRLMVSTKCGHLFCSQCIRDSLSRAHSCPTCRKKLTHKQYHPIYI

SEQ ID NO: 13 - protein, 185 aa, Unknown
  RING Domain
RKRKGAEPGHSKSSKRRAPGSTAAMTAATEPIELESGEEVVDLTCESTEPVVVDLTNNDLSINDSVVIVEDTPRQRRALSRPSQQTTSCVLSSDDEDSRHADHFAANKDISSQAYGSSRSSSGKVSCPICMDSYSEIVQSGRLIVSTKCGHIFCSQCLRDALKNAPSCPTCRKKLNHKQYHPIYV

SEQ ID NO: 14 - protein, 66 aa, Unknown
  RING Domain
SSSGKVSCPICMDSYSEIVQSGRLIVSTKCGHIFCSQCLRDALKNAPSCPTCRKKLNHKQYHPIYV

SEQ ID NO: 15 - protein, 185 aa, Unknown
  RING Domain
RKRKGAEPGLSKSRKRRAPGSTATMAAETEAIELESGEEVVDLTCESTEPVVVDLTNNDLSINDSVVIVEDTPRQRRTVSRTSHQTSSCVLSSDDEGSRDTELFATNKDISSPGYGSSRSSSGKVSCPICMDSYSEIVQSRRLIVSTKCGHIFCSQCLRDALKNALSCPTCRKKLNNKQYHPIYV

SEQ ID NO: 16 - protein, 66 aa, Unknown
  RING Domain
SSSGKVSCPICMDSYSEIVQSRRLIVSTKCGHIFCSQCLRDALKNALSCPTCRKKLNNKQYHPIYV

SEQ ID NO: 17 - protein, 176 aa, Unknown
  RING Domain
REGGPTPNIALDAELIELGESDEVVDLTCESLAPVVIDLTHRDSVVIIEQRRRPRANTRPLQDHTGSCVVNNDEEGVKRDRDVSTNPSSNIDPGNYVEMNDSITHLPSKVVIQDITMELHCPLCNDWFRDPLMLSCGHNFCEACIQDFWRLQAKETFCPECKMLCQYNNCTFNPVL

SEQ ID NO: 18 - protein, 194 aa, Rattus norvegicus
MSTRNPQRKRRGGAVNSRQTQKRTRETTSTPEISLEAEPIELVETVGDEIVDLTCESLEPVVVDLTHNDSVVIVEERRRPRRNGRRLRQDHADSCVVSSDDEELSKDKDVYVTTHTPRSTKDEGTTGLRPSGTVSCPICMDGYSEIVQNGRLIVSTECGHVFCSQCLRDSLKNANTCPTCRKKINHKRYHPIYI

SEQ ID NO: 19 - protein, 120 aa, Rattus norvegicus
EERRRPRRNGRRLRQDHADSCVVSSDDEELSKDKDVYVTTHTPRSTKDEGTTGLRPSGTVSCPICMDGYSEIVQNGRLIVSTECGHVFCSQCLRDSLKNANTCPTCRKKINHKRYHPIYI

SEQ ID NO: 20 - protein, 64 aa, Rattus norvegicus
SGTVSCPICMDGYSEIVQNGRLIVSTECGHVFCSQCLRDSLKNANTCPTCRKKINHKRYHPIYI

SEQ ID NO: 21 - protein, 61 aa, Unknown
  RING domain
QKEELYKCPICMDSVSKREPVSTKCGHVFCRECIETAIRATHKCPICNKKLTARQFFRIYL

SEQ ID NO: 22 - protein, 62 aa, Unknown
  RING Domain
PTPASVNCPICFESVYRRQAASTICGHLFCNACITAEMRIRKKCPLCKHPLKWQQVHPIYFN

SEQ ID NO: 23 - protein, 63 aa, Unknown
  RING Domain
VEEPKFSCPICLCPFTQEVSTKCGHIFCKKCIKNALSLQAKCPTCRKKITVKDLIRVFLPTTR

SEQ ID NO: 24 - protein, 64 aa, Unknown
  RING Domain
QRLADYKCVICLDSPENLSCTPCGHIFCNFCILSALGTTAATQKCPVCRRKVHPNKVICLEMML

SEQ ID NO: 25 - protein, 65 aa, Unknown
  RING Domain
GAAKDYRCPICFEPPETALMTLCGHVFCCPCLFQMVNSSRTCRQFGHCALCRSKVYLKDVRLIIL

SEQ ID NO: 26 - protein, 65 aa, Unknown
  RING Domain
TLEDIPVCCLCGAELGLSKRTFIASCGHAFCGRCFARIDYGPKLCPADSCKKLIRSRGRLKEVYF

SEQ ID NO: 27 - protein, 66 aa, Unknown
  RING Domain
HPHNNIACAKCGNELVSDEKKSIFAAKCGHLFCSTCAKELRKKTVPCPVQHCRKRITKKFIFPLYL

SEQ ID NO: 28 - protein, 520 aa, Unknown
  U-box domain
MGKRQHQKDKMYITCAEYTHFYGGKKPDLPQTNFRRLPFDHCSLSLQPFVYPVCTPDGIVFDLLNIVPWLKKYGTNPSNGEKLDGRSLIKLNFSKNSEGKYHCPVLFTVFTNNTHIVAVRTTGNVYAYEAVEQLNIKAKNFRDLLTDEPFSRQDIITLQDPTNLDKFNVSNFYHVKNNMKIIDPDEEKAKQDPSYYLKNTNAETRETLQELYKEFKGDEILAATMKAPEKKKVDKLNAAHYSTGKVSASFTSTAMVPETTHEAAAIDEDVLRYQFVKKKGYVRLHTNKGDLNLELHCDLTPKTCENFIRLCKKHYYDGTIFHRSIRNFVIQGGDPTGTGTGGESYWGKPFKDEFRPNLSHTGRGILSMANSGPNSNRSQFFITFRSCAYLDKKHTIFGRVVGGFDVLTAMENVESDPKTDRPKEEIRIDATTVFVDPYEEADAQIAQERKTQLKVAPETKVKSSQPQAGSQGPQTFRQGVGKYINPAATKRAAEEEPSTSATVPMSKKKPSRGFGDFSSW

SEQ ID NO: 29 - protein, 74 aa, Unknown
  U-box domain
RRLPFDHCSLSLQPFVYPVCTPDGIVFDLLNIVPWLKKYGTNPSNGEKLDGRSLIKLNFSKNSEGKYHCPVLFT

SEQ ID NO: 30 - RNA, 19 nt, Artificial Sequence
  siRNA duplexes
gaucacgucaguuucauca

SEQ ID NO: 31 - RNA, 19 nt, Artificial Sequence
  siRNA duplexes
ugaguuacauggacagucu

SEQ ID NO: 32 - RNA, 19 nt, Artificial Sequence
  siRNA duplexes
ccaacagucaguuucauga

SEQ ID NO: 33 - RNA, 19 nt, Artificial Sequence
  siRNA duplexes
gggauguacgugauaugua

SEQ ID NO: 34 - protein, 19 aa, Unknown
  NEDD8
TLTGKEIEIDIEPTDKVER

SEQ ID NO: 35 - protein, 14 aa, Unknown
  NEDD8
EIEIDIEPTDKVER

SEQ ID NO: 36 - protein, 17 aa, Unknown
  NEDD8
IKERVEEKEGIPPQQQR

SEQ ID NO: 37 - protein, 13 aa, Unknown
  NEDD8
VEEKEGIPPQQQR

SEQ ID NO: 38 - protein, 14 aa, Unknown
  NEDD8
ILGGSVLHLVLALR

SEQ ID NO: 39 - protein, 7 aa, Unknown
  NEDP1
LAFVEEK

SEQ ID NO: 40 - protein, 7 aa, Unknown
  NEDP1
LEAFLGR

SEQ ID NO: 41 - protein, 12 aa, Unknown
  NEDP1
QVAEKLEAFLGR

(19) United States
(12) Patent Application Publication
(10) Pub. No.: US 2024/0263151 A1
(43) Pub. Date: Aug. 8, 2024

(54) PROTEIN DEGRADATION

(71) Applicant: UNIVERSITY OF DUNDEE, Dundee (GB)

(72) Inventors: Ronald T. HAY, Dundee (GB); Adel F.M. IBRAHIM, Dundee (GB); Linnan SHEN, Dundee (GB)

(21) Appl. No.: 17/920,549

(22) PCT Filed: Apr. 21, 2021
(86) PCT No.: PCT/EP2021/060453

§ 371 Date: Oct. 21, 2022

(30) Foreign Application Priority Data
Apr. 22, 2020 (GB) 2005876.4

(51) Int. Cl. C12N 9/10; C07K 16/00

(52) U.S. Cl. CPC C12N 9/104; C07K 16/00; C07K 2317/31; C07K 2317/569; C07K 2317/76; C07K 2319/09

(57) ABSTRACT

The present disclosure provides a novel molecule or construct which can mediate or induce protein degradation. The molecules can be provided in the form of constructs and used to mediate the degradation of specific proteins. The molecules comprise an E3 ligase component and a target protein-binding moiety; this ensures the molecules can be tailored to the degradation of a specific proteinaceaous target.

### FIELD

The present disclosure provides molecules with an ability to degrade proteins, methods of making such molecules and compositions, uses and methods exploiting the same.

### BACKGROUND

Understanding the function of a gene usually requires ablation of expression of the gene product. In traditional genetic and more recent genome editing (Doudna and Charpentier, 2014) approaches, changes to the genetic material result in inactivation, ablation of expression or alteration of the activity of the gene product that manifest themselves in an altered phenotype, that is presumed to be directly linked to the function of the protein. Alternative methods such as RNA interference (RNAi) lead to destruction of the mRNA but have no direct effect on the protein product of the gene (Elbashir et al., 2001) which is thus depleted at a rate defined by the inherent half-life of the protein. This precludes the use of such approaches to remove proteins with a very long half-life (Toyama et al., 2013) or insoluble protein aggregates that are typically associated with neurological disease. RNAi based approaches also have the disadvantage of taking a long time to deplete protein levels (typically 48 hrs). This is particularly troublesome when studying processes like the cell cycle, where protein depletion is only achieved after multiple cell cycles. Such delays in protein depletion also give the cell time to initiate compensatory mechanisms which may mask the primary phenotype of target protein depletion.

To directly induce degradation of a protein of interest a number of approaches have harnessed the power and specificity of the protein degradation machinery of the cell. Proteins are first targeted for ubiquitination and the ubiquitinated proteins are destroyed by the proteasome (Hershko and Ciechanover, 1998). Ubiquitin E3 ligases are the molecules that recognise substrates and mediate their ubiquitination. Most methods either artificially target the protein to a pre-existing ubiquitin E3 ligase or generate new E3 ligases engineered to recognise particular proteins. Proteolysis Targeting Chimeras (PROTACs) are bifunctional chemical entities that can bind to target proteins and recruit them to a pre-existing ubiquitin E3 ligase (Bondeson et al., 2015; Runcie et al., 2016; Sakamoto et al., 2001). This approach has the advantage that endogenous proteins can be targeted and the cell does not have to be modified. However the protein to be targeted must bind with high affinity to a ligand contained within the PROTAC molecule, and such ligands do not exist for most proteins.

A method that allows rapid, ligand-induced degradation of target proteins is the Auxin Inducible Degron (AID) system (Holland et al., 2012; Nishimura et al., 2009). In this approach non-plant cells are engineered to express the plant ubiquitin E3 ligase TIR1 that is inactive until it binds to the plant hormone auxin. In its active, auxin-bound state it recognises a specific protein sequence, known as a degron, that can be engineered into a protein to be targeted for degradation. In the absence of auxin the protein is stable, but undergoes rapid degradation when auxin is added to the medium of the cells. While this approach enables rapid degradation of the target protein it necessitates engineering of the cells to express plant TIR1 and a degron tagged protein target.

While all of these methods have particular advantages and disadvantages, none represents a system which is based on a single component that could be produced in high yield and used as a reagent that could be introduced into cells and induce the rapid and specific degradation of endogenous proteins.

### SUMMARY

The present disclosure provides a novel molecule or construct which can mediate or induce protein degradation. The molecules described herein may find particular application as molecules which are able to mediate the degradation of specific proteins—referred to hereinafter as ‘target proteins’.

The innovation described herein provides a single molecule which can quickly and easily be replicated or manufactured in high yield systems. The inventors have also found that the disclosed molecules can be easily introduced into cells, optionally as genetic constructs and induced to express themselves. Accordingly, the molecules have both in vitro and intracellular (in vivo) uses.

Moreover they have been found to act rapidly and specifically allowing efficient and targeted degradation of any predetermined, specific (targeted) protein.

A further advantage associated with the molecules described herein is that they yield no/low (or substantially no) observable off target effects; that is to say they are highly specific.

Accordingly, this disclosure not only provides said molecules/construct but also compositions and medicaments comprising the same, methods of treatment using the disclosed compositions and medicaments, methods of making and using these molecules/constructs and various kits.

It should be noted that the terms “comprise”, “comprising” and/or “comprises” is/are used to denote that aspects and embodiments of this disclosure “comprise” a particular feature or features. It should be understood that this/these terms may also encompass aspects and/or embodiments which “consist essentially of” or “consist of” the relevant feature or features.

This disclosure provides a molecule which may be used to degrade proteins.

The molecules of this disclosure may be referred to as “constructs”—that is a manufactured or synthetic molecule made by the modification of some protein sequence, for example a wild-type protein sequence. Modifications may include the mutation (by addition, deletion or inversion) of certain residues of the protein sequence. A molecule of this disclosure may also be constructed by the joining of one protein sequence to another to create a protein fusion (a ‘fusion’).

A molecule of this disclosure may be derived from an ubiquitin E3 ligase.

A molecule of this disclosure may comprise an E3 ligase component and a target protein binding moiety.

The E3 ligase component may be fused (optionally via a short linker molecule) to the target protein binding moiety.

The E3 ligase component may function to recruit the ubiquitin loaded E2 conjugating enzyme.

In one embodiment, the molecule may comprise (i) a molecule which recruits or binds the ubiquitin loaded E2 conjugating enzyme and (ii) a target protein binding moiety. The molecule may comprise a fusion between a molecule which recruits or binds the ubiquitin loaded E2 conjugating enzyme and a target protein binding moiety.

Without wishing to be bound by theory, a molecule of this disclosure may facilitate the degradation of a target protein by binding that protein (via the target protein binding moiety part) and recruiting the ubiquitin loaded E2 conjugating enzyme so as to transfer ubiquitin to the target protein. This leads to degradation of the target protein via the proteasome.

A molecule of this disclosure may be derived from a SUMO-targeted ubiquitin ligase—this being a member of the ubiquitin E3 ligase family.

Without wishing to be bound by any particular theory, the covalent and posttranslational modification of a protein with a small ubiquitin-related modifier (SUMO) is a mechanism by which the function of an array of cellular proteins is regulated/modulated. The SUMO-targeted ubiquitin ligases (a class of ubiquitin E3 ligases) recognise sumoylated proteins and the concurrent recruitment of the ubiquitin loaded E2 conjugating enzyme leads to the transfer of ubiquitin to the substrate. This leads to degradation of the substrate via the proteasome.

By changing the substrate recognition properties of the SUMO-targeted ubiquitin ligase it is possible to provide molecules which are active against defined and/or predetermined targets.

A molecule of this disclosure may comprise a modified SUMO-targeted ubiquitin ligase.

A SUMO-targeted ubiquitin ligase may be modified by removal, ablation or replacement of one or more of the SUMO recognition domains of a SUMO-targeted ubiquitin ligase.

A SUMO-targeted ubiquitin ligase may be modified by replacement of one or more of the SUMO recognition domains by or with a moiety which binds to, or associates with, a target protein.

A molecule of this disclosure may comprise a RING domain sequence derived or obtained from an ubiquitin E3 ligase molecule. An exemplary RING domain sequence may be derived from a SUMO-targeted ubiquitin ligases.

A molecule of this disclosure may further or additionally comprise a moiety which binds to, or associates with, a target protein.

Accordingly, this disclosure provides a molecule comprising:
(i) an E3 ligase component and a target protein binding moiety; or (ii) the RING domain of a SUMO-targeted ubiquitin ligase and
a target protein binding moiety.

The E3 ligase component may be any E3 ligase derived component which recruits ubiquitin-loaded E2 enzyme. By way of example, useful E3 ligase components may comprise RING-like domains such as U-boxes—which recruit the ubiquitin-loaded E2.

Suitable U-box proteins may be derived from, for example, the E4B ubiquitin ligase. The E4B enzyme is a U-box-containing protein that functions as an E3 ubiquitin ligase.

Accordingly, a molecule of this disclosure may comprise a U-box moiety and a target protein binding moiety. The U-box moiety may be fused (optionally via a short linker) to the target protein binding moiety.

The molecule, or at least the RING domain of a SUMO-targeted ubiquitin ligase, may not comprise one or more of the SUMO recognition domains usually present in a SUMO-targeted ubiquitin ligase

Useful RING domains may comprise the C-terminal RING domain of a SUMO-targeted ubiquitin ligase. Among the functions assigned to these RING domains are an ability to recruit the ubiquitin loaded E2 conjugating enzyme.

The RING-domain may be derived from the ubiquitin E3 ligase RNF4.

Accordingly, the disclosure provides a molecule comprising:
the RING domain of ubiquitin E3 ligase RNF4 and
a target protein binding moiety.

In one embodiment, the RING domain is not a RING domain from a LNX1 protein.

Useful RING domains may comprise or be obtained from any of SEQ ID NOS: 1-27 reproduced below. These sequences comprise the C-terminal RING domain of a SUMO-targeted ubiquitin ligase. Again, among the functions assigned to these RING domains are an ability to recruit the ubiquitin loaded E2 conjugating enzyme.

It should be noted that a RING domain is defined by the presence of 7 Cysteine and 1 histidine residues. This motif is embedded in additional sequences which make up the fold of the RING. Accordingly, the full and useful RING domain sequence does not necessarily have a defined sequence. The skilled reader will therefore understand that the precisely start position of a useful RING sequence may vary (by for example ±1, 2, 3, 4 or 5 residues)—this variation may apply to the highlighted (underlined) sequences below) which are intended to serve as indicative RING domain sequences only.

SEQ ID NO: 1
RKRRGGAINSRQAQKRTREATSTPEISLEAEPIELVETAGDEIVDLTCESLEPVVVDLTHNDSVVIVD
ERRRPRRNARRLPQDHADSCVVSSDDEELSRDRDVYVTTHTPRNARDEGATGLRPSGTVSCPICMDGY
SEIVQNGRLIVSTECGHVFCSQCLRDSLKNANTCPTCRKKINHKRYHPIYI
The RING domain sequence of SEQ ID NO: 1 is underlined. That sequence may be
provided as SEQ ID NO: 2.
SEQ ID NO: 3
RKRRGGTVNSRQTQKRTRETTSTPEVSLETEPIELVETVGDEIVDLTCESLEPVVVDLTHNDSVVIVE
ERRRPRRNGRRLRQDHADSCVVSSDGEELSRDKDVYVTTHTPRSTKDDGATGPRPSGTVSCPICMDGY
SEIVQNGRLIVSTECGHVFCSQCLRDSLKNANTCPTCRKKINHKRYHPIYI
The RING domain sequence of SEQ ID NO: 3 is underlined. That sequence may be
provided as SEQ ID NO: 4.
SEQ ID NO: 5
RKRRGGSANSRQAQKRSRLIASTTEMASEGEPIELEESAGEEVVDLICESSDPVVVDLTHNDSIVIVE
ENQQRRRNLRLRGQRQSDSCVLSSDDEDETRDNDVYVTDKVSRELGPLEDETASSKPSGTVSCPICMD
GYSEIVQSGRLIVSTKCGHVFCSQCLRGSLRNANSCPTCRKKLTHRQYHPIYI
The RING domain sequence of SEQ ID NO: 5 is underlined. That sequence may be
provided as SEQ ID NO: 6.
SEQ ID NO: 7
RKRRSGTTNSRQAQKRERLVGPTSDMTSETEPIELVESAGEEVVDLTCESTEPVVVDLTHNDPVVFIE
ENRRQRRNPRISRGQPDSCVLSSDDEEIRDNDVYVTTENPSRESPDPAGFGEKPSGTVSCPICMDGYS
EILQSGRLIVSTKCGHVFCSQCLRDALRNASSCPTCRKKLNQKQYHPIYI
The RING domain sequence of SEQ ID NO: 7 is underlined. That sequence may be
provided as SEQ ID NO: 8.
SEQ ID NO: 9
GSMVNSRQAREFHTAGERGPTPQTVLEAELIELGESDEVVDLICESLEPAVIDLTHQDCVVITEERRR
PRGNTRSLQGQTGSCVVSSDKELMRDRDVYVTHSAYHNALEEETLSCTLPGYIQCRICMDGYSEIELS
RRHIYSTDCGHIFCSQCLCTSLKYTKTCPVCLKKIGCRQYHRIYL
The RING domain sequence of SEQ ID NO: 9 is underlined. That sequence may be
provided as SEQ ID NO: 10.
SEQ ID NO: 11
VTQRKRRISTTCSRRGNSKRNRAQMSQTVMETIDVLENDRINSEDVVDLTCEGSEPAVVDLINNDSIV
VVEDGVQRRVGPCTESYVLSSDEEEESSLRLVYVTSPGLLSSLRDSSRARSTGAISCPVCMDVYSEIM
DSGRLMVSTKCGHLFCSQCIRDSLSRAHSCPTCRKKLTHKQYHPIYI
The RING domain sequence of SEQ ID NO: 11 is underlined. That sequence may be
provided as SEQ ID NO: 12.
SEQ ID NO: 13
RKRKGAEPGHSKSSKRRAPGSTAAMTAATEPIELESGEEVVDLICESTEPVVVDLINNDLSINDSVVI
VEDTPRQRRALSRPSQQTTSCVLSSDDEDSRHADHFAANKDISSQAYGSSRSSSGKVSCPICMDSYSE
IVQSGRLIVSTKCGHIFCSQCLRDALKNAPSCPTCRKKLNHKQYHPIYV
The RING domain sequence of SEQ ID NO: 13 is underlined. That sequence may be
provided as SEQ ID NO: 14.
SEQ ID NO: 15
RKRKGAEPGLSKSRKRRAPGSTATMAAETEAIELESGEEVVDLTCESTEPVVVDLINNDLSINDSVVI
VEDTPRQRRTVSRTSHQTSSCVLSSDDEGSRDTELFATNKDISSPGYGSSRSSSGKVSCPICMDSYSE
IVQSRRLIVSTKCGHIFCSQCLRDALKNALSCPTCRKKLNNKQYHPIYV
The RING domain sequence of SEQ ID NO: 15 is underlined. That sequence may be
provided as SEQ ID NO: 16.
SEQ ID NO: 17
REGGPTPNIALDAELIELGESDEVVDLTCESLAPVVIDLTHRDSVVIIEQRRRPRANTRPLQDHTGSC
VVNNDEEGVKRDRDVSTNPSSNIDPGNYVEMNDSITHLPSKVVIQDITMELHCPLCNDWFRDPLMLSC
GHNFCEACIQDFWRLQAKETFCPECKMLCQYNNCTENPVL
An exemplary RING finger protein 4 sequence is the Rattus norvegicus RNF4 sequence
(accession: NM_019182, UniProtKB-O88846). This sequence is reproduced as SEQ ID NO:
18 below:
SEQ ID NO: 18
MSTRNPQRKRRGGAVNSRQTQKRTRETTSTPEISLEAEPIELVETVGDEIVDLTCESLEPVVVDLTHN
DSVVIVEERRRPRRNGRRLRQDHADSCVVSSDDEELSKDKDVYVTTHTPRSTKDEGTTGLRPSGTVSC
PICMDGYSEIVQNGRLIVSTECGHVECSQCLRDSLKNANTCPTCRKKINHKRYHPIYI

A useful RING domain sequence may be derived from SEQ ID NO: 18. For example a sequence comprising (or consisting essentially of or consisting of) residues 75-194 (underlined residues: SEQ ID NO: 19) or residues 131-194 (double underlined residues: SEQ ID NO: 20) may provide a RING domain sequence for use in a molecule, method, composition or kit of this disclosure.

Additional SUMO-targeted ubiquitin ligase derived RING domain sequences may comprise the following sequences any of which may be used to make a molecule of this disclosure.

SEQ ID NO: 21
QKEELYKCPICMDSVSKREPVSTKCGHVFCRECIETAIRATHKCPICNK
KLTARQFFRIYL
SEQ ID NO: 22
PTPASVNCPICFESVYRRQAASTICGHLFCNACITAEMRIRKKCPLCKH
PLKWQQVHPIYFN
SEQ ID NO: 23
VEEPKFSCPICLCPFTQEVSTKCGHIFCKKCIKNALSLQAKCPTCRKKI
TVKDLIRVFLPTTR
SEQ ID NO: 24
QRLADYKCVICLDSPENLSCTPCGHIFCNFCILSALGTTAATQKCPVCR
RKVHPNKVICLEMML
SEQ ID NO: 25
GAAKDYRCPICFEPPETALMTLCGHVFCCPCLFQMVNSSRTCRQFGHCA
LCRSKVYLKDVRLIIL
SEQ ID NO: 26
TLEDIPVCCLCGAELGLSKRTFIASCGHAFCGRCFARIDYGPKLCPADS
CKKLIRSRGRLKEVYF
SEQ ID NO: 27
HPHNNIACAKCGNELVSDEKKSIFAAKCGHLFCSTCAKELRKKTVPCPV
QHCRKRITKKFIFPLYL

RING domain sequences derived from SUMO-targeted ubiquitin ligases are relatively small molecules—that is small (in terms of the total number of residues as compared to the size (again, in terms of the number of RING domain residues) of other RING domain sequences. Indeed the inventors have discovered that small fragments of the larger SUMO-targeted ubiquitin ligase are functional and can be used in the manufacture of molecules of this disclosure. The small size of the RING domain of a SUMO-targeted ubiquitin ligase (for example the RING domains of SEQ ID NOS: 1-27 above) means that molecules of this disclosure (which molecules may comprise a RING domain of a SUMO-targeted ubiquitin ligase) may easily be expressed in recombinant systems (for example bacteria); this allows large amounts of (recombinant) material to be produced. There is also the added advantage that the small size of the RING domain of a SUMO-targeted ubiquitin ligase allows it to be introduced into cells by, for example, electroporation, liposomes and the like. The molecules (or nucleic acids encoding the same) may also be transfected.

The molecules of this disclosure may also be used without the need to induce expression within a cell—in other words, the protein may be added directly to a cell and will work (to degrade proteins) without any induction. The molecules of this disclosure may work in a non-inducible manner.

The molecules of this disclosure may be ready (and readily) assembled, exogenously expressed. The molecules may form part of a non-inducible protein targeting system which utilises a cell's internal degradation machinery (the ubiquitin-proteasome system).

It should be noted that a RING domain sequence for use in a molecule, method, composition or kit of this disclosure may comprise about 130, about 120, about 110, about 100, about 95, about 90, about 85, about 80, about 75, about 70, about 69, about 68, about 67, about 66, about 65, about 64, about 63, about 62, about 61 or about 60 residues of any of the RING domain sequences described herein—including those presented as SEQ ID NOS: 1-27 above. Sequences of this type may be described as fragments of SEQ ID NOS: 1-27. Useful fragments may exhibit one or more (for example all) of the properties of the native RING domain sequence. For example a useful fragment may function to recruit the ubiquitin loaded E2 conjugating enzyme. The fragment may further facilitate dimerization (that is to say, dimerization of RING domains). These fragments may be referred to as “RING domain” fragments.

A RING domain sequence may comprise from about residue 70, 71, 72, 73, 74, 75, 76, 77, 78, 79 or 80 to about residue ‘n’, where ‘n’ is the last residue (the value of ‘n’ will vary depending on the total number of residues in the sequence).

In addition to the sequences provided above as SEQ ID NOS: 1-27, the disclosure may embrace other sequences with identity and/or homology to any of the SEQ ID NOS: 1-27 or to any of the RING domain fragments described above. For example, the disclosure may relate to sequences which have at least 99%, at least 98%, at least 97%, at least 96%, at least 95%, at least 94% at least 93% at least 92% at least 91% at least 90%, at least 85%, at least 80%, at least 75%, at least 70%, at least 65% or at least 60% sequence identity or sequence homology to (or with) any one of the sequences disclosed as SEQ ID NOS: 1-27 or any of the RING domain fragments described herein.

In view of the above, the disclosure provides a molecule comprising:
a sequence comprising any one of SEQ ID NOS: 1-27, or a RING domain fragment thereof; and
a target protein binding moiety.

A useful U-box sequence may be derived from the sequence deposited as A0A024R1C1 (interPro database). That sequence is reproduced below as SEQ ID NO: 28. The U-box domain is highlighted in underline—this sequence may be provided as SEQ ID NO: 29.

MGKRQHQKDKMYITCAEYTHFYGGKKPDLPQTNFRRLPFDHCSLSLQPF
VYPVCTPDGIVEDLLNIVPWLKKYGTNPSNGEKLDGRSLIKLNFSKNSE
GKYHCPVLFTVFTNNTHIVAVRTTGNVYAYEAVEQLNIKAKNERDLLTD
EPFSRQDIITLQDPTNLDKFNVSNFYHVKNNMKIIDPDEEKAKQDPSYY
LKNINAETRETLQELYKEFKGDEILAATMKAPEKKKVDKLNAAHYSTGK
VSASFTSTAMVPETTHEAAAIDEDVLRYQFVKKKGYVRLHTNKGDLNLE
LHCDLTPKTCENFIRLCKKHYYDGTIFHRSIRNFVIQGGDPTGTGTGGE
SYWGKPFKDEFRPNLSHTGRGILSMANSGPNSNRSQFFITFRSCAYLDK
KHTIFGRVVGGEDVLTAMENVESDPKTDRPKEEIRIDATTVFVDPYEEA
DAQIAQERKTQLKVAPETKVKSSQPQAGSQGPQTERQGVGKYINPAATK
RAAEEEPSTSATVPMSKKKPSRGFGDFSSW

As stated, a molecule of this disclosure may comprise an E3 ligase component which is derived from a U-box sequence; a useful U-box sequence may comprise that which is provided by SEQ ID NO: 28 or functional fragment thereof. A functional fragment may comprise any fragment of SEQ ID NO: 28 which is able to recruit the ubiquitin loaded E2 conjugating enzyme. One useful fragment may comprise the sequence provided by SEQ ID NO: 29. Similar fragments may be referred to as U-box domain fragments.

It should be noted that a U-box sequence for use in a molecule, method, composition or kit of this disclosure may comprise about from about 50 to (a-1) residues of SEQ ID NO: 28—where ‘a’ is the total number of residues (520) in SEQ ID NO: 28. A U-box sequence for use in a molecule, method, composition or kit of this disclosure may comprise about 60, about 65, about 70, about 71, about 72, about 73, about 74, about 75, about 76, about 77, about 78, about 79, about 80, about 85, about 90, about 95, about 100, about 150, about 200, about 250, about 300, about 350, about 400, about 450, about 500, about 505, about 510, about 515, about 516, about 517, about 518, about 519 or about 520 residues of any suitable U-box sequence including that provided by SEQ ID NO: 28 above.

A U-box domain sequence may comprise from about 65, about 66, about 67, about 68, about 69, about 70, about 71, about 72, about 73, about 74, about 75, about 76, about 77, about 78, about 79 or about 80 residues of SEQ ID NO: 28.

In addition to the sequences provided above as SEQ ID NOS: 28-29, the disclosure may embrace other sequences with identity and/or homology to any of the SEQ ID NOS: 28-29 or to any of the U-box domain fragments described above. For example, the disclosure may relate to sequences which have at least 99%, at least 98%, at least 97%, at least 96%, at least 95%, at least 94% at least 93% at least 92% at least 91% at least 90%, at least 85%, at least 80%, at least 75%, at least 70%, at least 65% or at least 60% sequence identity or sequence homology to (or with) any one of the sequences disclosed as SEQ ID NOS: 28-29 or any of the U-box domain fragments described herein.

In view of the above, a molecule of this disclosure may comprise a sequence comprising SEQ ID NOS: 28, 29 or a functional fragment thereof and a target protein binding moiety.

As stated, a functional fragment may comprise a fragment which recruits the ubiquitin loaded E2 conjugating enzyme.

The E3 ligase component (which includes any of the E3 ligase components described herein, including any RING domain of a SUMO-targeted ubiquitin ligase, any E3 ligase component which functions to recruit the ubiquitin loaded E2 enzyme, U-Box type RING domains and/or the RNF4 E3 ligase (or the RING domain thereof)) may comprise or be derived from a “modified E3 ligase”. A modified E3 ligase may include one or more amino acid mutations relative to a reference (or wild-type) E3 ligase sequence. By way of example, relative to a reference sequence, a modified E3 ligase component may comprise one or more amino acid substitutions, additions, deletions and/or inversions. By way of non-limiting example, a useful modified E3 ligase component may comprise (relative to a reference E3 ligase sequence) one or more conservative amino acid substitutions. A modified E3 ligase may function to recruit ubiquitin-loaded E2 enzyme.

In one teaching, a modified E3 ligase may be modified by substitution of one or more lysine residues (in the wild type E3 ligase sequence) with arginine residues. This would constitute a conservative substitution of the wild-type lysine residue.

Without wishing to be bound by theory, a modified E3 ligase may be less vulnerable/susceptible to autoubiquitination (where the E3 ligases component is itself ubiquitinated and becomes degraded. In the absence of a substrate, this process may be elevated in the absence of substrate. Again, without wishing to be bound by theory, it is thought that this autoubiquitination process might serve as a means for removing excess or unwanted E3 ligase.

The phenomenon of autoubiquitination may take place on lysine residues and therefore and without being bound to the theory, it may be possible to increase the stability of the E3 ligase by substituting some or all of the lysine residues in the E3 ligase to arginine. This (conservative) mutation may retain a positive charge on the protein but since arginine residues cannot be ubiquitinated, the modified E3 ligase becomes more stable and persistent.

An E3 ligase that has had all of its lysine residues changed to arginine may be refractory to ubiquitination and degradation.

An advantage associated with some of the constructs/molecules described herein is that in contrast to some large E3 complexes which altogether may contain 100 lysine residues, the disclosed constructs/molecules may comprise E3 ligase elements with fewer lysine resides. As a consequence, the (conservative) replacement of up to all of the lysine residues with arginine has less of an impact on the overall function and/or performance of the E3 ligase component. For example, in the case of a fusion comprising a RNF4 RING (for example a GFP nanobody-RNF4 RING fusion (GNb-RING)) there may be as few as 10 lysine residues.

In view of the above, this disclosure provides a molecule comprising:
(i) a modified E3 ligase component and a target protein binding moiety; or (ii) a modified RING domain of a SUMO-targeted ubiquitin ligase and
a target protein binding moiety.

In one teaching a molecule of this disclosure may comprise a modified E3 ligase component in which one or more or all of the lysine (K) residues of the E3 ligase component have been substituted with arginine (R).

In another teaching a molecule of this disclosure may comprise a modified RING domain of a SUMO-targeted ubiquitin component in which one or more or all of the lysine (K) residues of the SUMO-targeted ubiquitin component have been substituted with arginine (R). The modified E3 ligase component may comprise a modified RING domain of RNF4. In a modified RNF4 RING domain, one or more of the lysine residues my be substituted for (replaced with) an arginine residue. For example, one or more of the following substitutions may be introduced: K151R and/or K153R and/or K166R and/or K217R and/or K227R and/or K228R and/or K232R.

The target binding moiety (for use in any of the molecules described herein) may be any moiety which binds to and/or associates with a target protein.

A target protein may be a protein (or peptide) which is to be degraded.

A target protein may be a protein which is endogenous to a cell (i.e. ‘an endogenous protein’).

The target protein may be a nuclear protein.

The target protein may be a cytosolic protein.

The target protein may be a soluble protein and/or in solution.

The target protein may be insoluble or present as inclusion bodies, aggregates, nuclear bodies and the like.

The target protein may be associated with a disease or condition.

The target protein may be a disease causing protein.

The target protein may not be the active portion of a toxin (for example a microbial and/or bacterial toxin). The target protein may not be a toxin, for example a microbial and/or bacterial toxin.

The target protein binding moiety may be an antibody with specificity and/or affinity for the target protein.

The target protein binding moiety may be an antibody with specificity and/or affinity for one, two or more target protein(s).

The target protein binding moiety may be, for example, bi-specific, that is to say it is capable of binding to two different target proteins. For example the target protein binding moiety may bind an extracellular/cell surface protein and an intracellular protein.

The target protein binding moiety may be a bi-specific antibody.

The term ‘antibody’ may include any target protein binding fragment thereof.

The term ‘antibody’ may include, for example:
F(ab′)2 fragments (these fragments being characterised by lacking most (but perhaps not all) of the Fc portion and two antigen (or target protein) binding regions linked by disulphide bridges);
Fab′ fragments (this may be derived from a F(ab′)2; the fragment comprises one constant and one variable domain of each of the heavy and the light chains. The fragment may contain a small part of the Fc portion);
Fv fragments (including single chain (sc) Fv fragments: these fragments are characterised as fusion proteins of the variable regions of the immunoglobulin heavy and light chains connected with linker peptides)

The term ‘antibody’ may also include, for example, those molecules referred to as a ‘single-domain antibody’ (sdAb) or ‘nanobody’. These molecules comprise a single monomeric variable antibody domain. It is able to bind selectively to a specific antigen. Typically, these molecules have a low molecular weight of only 12-15 kDa and are thus much smaller than ‘normal’ antibodies (which may be of the order of 150-160 kDa in size). Nanobodies (or sdAb) are also smaller than Fab fragments and single-chain variable fragments.

It should be noted that the terms “antibody” and/or “nanobody” embrace bi-specific nanobodies.

It should also be noted that the terms “target protein binding moiety”, “antibody” and/or “nanobody”, include modified target protein binding moieties. A modified target protein-binding moiety may include one or more amino acid mutations relative to a reference (or wild-type) target protein binding moiety sequence. By way of example, relative to a reference sequence, a modified target protein-binding moiety may comprise one or more amino acid substitutions, additions, deletions and/or inversions. By way of non-limiting example, a useful modified target protein-binding moiety may comprise (relative to a reference sequence) one or more conservative amino acid substitutions. Any modified target protein-binding moiety should function to bind the target protein.

In one teaching, a modified target protein-binding moiety may be modified by substitution of one or more lysine residues (in the wild type modified target protein binding moiety sequence) with arginine residues. This would constitute a conservative substitution of the wild-type lysine residue.

As stated with reference to the potential use of modified E3 ligases, a modified target protein-binding moiety may be less vulnerable/susceptible to autoubiquitination (where the E3 ligases component is itself ubiquitinated and becomes degraded.

A target protein-binding moiety that has been modified by having had all of its lysine residues changed to arginine may be refractory to ubiquitination and degradation.

In view of the above, this disclosure provides a molecule comprising:
(i) an E3 ligase component or modified E3 ligase component; and
(ii) a target protein binding moiety or a modified target protein binding moiety.

In one teaching, a molecule of this disclosure may comprise a modified E3 ligase component and/or a modified target protein-binding moiety, wherein one or more or all of the lysine (K) residues of the E3 ligase component/target protein-binding molecule have been substituted with arginine (R). In one teaching, the target protein binding moiety may comprises a nanobody, the sequence of which has been modified to substitute one or more of its lysine residues with arginine residues. A ‘modified target protein binding moiety of this type may be combined with an E3 ligase component which has been modified in the same way (i.e. comprising one of more K/R mutations).

Useful nanobodies/sdAb may be obtained by immunising dromedaries, camels, llamas, alpacas or sharks with the desired antigen and subsequent isolation of the mRNA coding for heavy-chain antibodies. For example, to generate a target protein binding moiety for use with molecules described herein, a suitable animal (for example a dromedary, a camel, a llama, an alpaca or a shark) may be immunised with the target protein (or any immunogenic fragment thereof) so as to induce the generation of nanobodies with specificity or affinity for the target protein (the target protein being any protein which is to be degraded).

Alternatively single domain antibodies can be obtained from synthetic phage display libraries.

It should be noted that it is possible to generate useful nanobodies (or sdAb) from murine or human immunoglobulins.

The term “antibody” may embrace a camelid nanobody with specificity for a target protein.

The term “antibody” may embrace a camelid bi-specific nanobody with specificity for at least two target proteins.

Accordingly, the disclosure provides a molecule comprising:
(i) an E3 ligase component and a target protein binding nanobody; or
(ii) the RING domain of a SUMO-targeted ubiquitin ligase and
a target protein binding nanobody.

Moreover, the disclosure provides a molecule comprising:
(i) an E3 ligase component and a target protein binding camelid nanobody; or
(ii) the RING domain of a SUMO-targeted ubiquitin ligase and
a target protein binding camelid nanobody.

The disclosure also provides a molecule comprising:
(i) an E3 ligase component and a green fluorescent protein (GFP) binding moiety; or
(ii) the RING domain of a SUMO-targeted ubiquitin ligase and
a green fluorescent protein (GFP) binding moiety.

The GFP binding moiety may comprise a GFP binding nanobody. An exemplary GFP binding nanobody may be a camelid-derived single-domain antibody with specificity for GFP.

An exemplary GFP binding moiety may comprise the camelid-derived single-domain nanobody deposited as 3K1K_C (PDB accession: deposited 28 Sep. 2009: Kirchhofer, A et al).

A molecule comprising a GFP binding moiety is useful as such molecules can be used where a binding moiety (for example a nanobody) with specificity for a target protein is not available. In such cases, if the target protein can be presented or made as a GFP-tagged or fused protein, then a molecule with a GFP-binding moiety could be used to effect degradation of that GFP-tagged/fused protein.

A molecule of this disclosure may comprise two or more RING domains fused to a target protein binding moiety. A molecule of this type may be described as ‘constitutively dimeric’.

A constitutively dimeric form of a molecule of this disclosure may comprise:
(a) two RING domains of a SUMO-targeted ubiquitin ligase and
a target protein binding moiety; or
(b) two sequences comprising any one of SEQ ID NOS: 1-27, or a RING domain fragments thereof and

a target protein binding moiety.

In a constitutively dimeric molecule, the two RING domains of a SUMO-targeted ubiquitin ligase may be joined by a short linker molecule.

The molecules of this disclosure may further comprise the nuclear localisation signal (NLS) of the RING domains of a SUMO-targeted ubiquitin ligase. In this way, the molecule may be able to efficiently target nuclear proteins.

The disclosure provides an inducible construct comprising (i) an E3 ligase component and a target protein binding moiety; or (ii) the RING domain of a SUMO-targeted ubiquitin ligase and a target protein binding moiety.

More specifically the disclosure provides an inducible construct of (i) the E3 ligase component and a target protein binding nanobody; or (ii) the RING domain of the ubiquitin E3 ligaseRNF4 and a camelid nanobody with specificity or affinity for a target protein.

As stated, these molecules (namely all of the molecules described herein) are able to mediate the destruction of a target protein by the ubiquitin proteasome system.

The target protein may be associated with a diseases and/or pathological condition. For example, the target protein may be a protein which when over expressed is associated with some sort of pathology or disease. The target protein may be the product of an oncogene.

It should be noted that in the case of a bi-specific target protein binding moiety, one of the target proteins may be a protein is to be bound by a molecule of this disclosure, but not necessarily degraded. For example, the target binding protein of the molecule may be designed to bind an extracellular protein, for example a cell surface protein or protein within the extracellular matrix. One of skill will appreciate that by binding to target proteins of this type (extracellular/cell surface/matrix proteins and the like) a molecule of this disclosure may be internalised and delivered into a cell. In the cell, the target protein binding moiety may target a second protein which is then degraded via the intracellular ubiquitin/proteasome system.

The target protein may be a mutated protein—that is a protein which contains, relative to a reference sequence, one or more mutations. A reference sequence may comprise, for example the wild-type sequence of a particular protein and a mutated form of that sequence may include one or more amino acid mutations (the addition, deletion or inversion of one or more amino acid residues).

The disclosure further provides nucleic acid sequences which encode or provide the molecules described herein.

These nucleic acid sequences may comprise DNA, cDNA or RNA.

The nucleic acid sequences may be for introduction into a cell.

The nucleic acid sequences may be inducible sequences—that is, once introduced into a cell their expression can be induced.

The nucleic acid sequences may be provided in the form of a vector, for example a plasmid.

Accordingly, the disclosure provides vectors comprising a nucleic acid sequence encoding any of the molecules described herein. For example a vector (plasmid) of this disclosure may comprise a nucleic acid sequence encoding any one of SEQ ID NOS: 1-27 described herein or a RING domain fragment thereof. A vector (for example plasmid) may additionally include a nucleic acid sequence encoding a target protein binding moiety as described herein. The nucleic acid sequence may encode a nanobody specific for a particular target protein.

The disclosure further provides a host cell transformed with a vector described above.

The molecules described herein may find application in (methods for) the degradation of proteins.

The term ‘degradation’ may relate to the breakdown or disintegration of a protein into smaller amino acid or peptide units. The degradation of a protein may destroy or ablate its function. The term “degradation” as used herein embraces degradation via the ubiquitin system (i.e. the ubiquitin-proteasome system). That is to say, a molecule of this disclosure can be used to degrade a target protein via the ubiquitin-proteasome system.

Thus, the disclosure provides the use of a molecule comprising:
(a) an E3 ligase component and a target protein binding moiety; or
(b) the RING domain of a SUMO-targeted ubiquitin ligase and
a target protein binding moiety; or
(c) a sequence comprising any one of SEQ ID NOS: 1-27, or a RING domain fragment thereof and
a target protein binding moiety; or
(d) a sequence comprising any one of SEQ ID NOS: 28-29, or a U-box domain fragment thereof; and
a target protein binding moiety;

for degrading proteins.

For the avoidance of doubt, any of the molecules described herein may be exploited in this use. Moreover, the proteins to be degraded are ‘target proteins’ as described herein.

Additionally, the disclosure provides a method of degrading a protein, said method comprising contacting a protein to be degraded with a molecule described herein. The method may comprise degrading a target protein of the type described herein.

It should be noted that the target protein may be an intracellular protein and therefore the molecules of this disclosure may be used to degrade intracellular (including cytosolic proteins and/or nuclear proteins). The target protein may be an unmodified, intracellular protein.

A method of degrading an intracellular protein with a molecule of this disclosure may comprise contacting a cell comprising a protein to be degraded, with a molecule of this disclosure.

The method may further comprise the step of contacting the cell with a molecule of this disclosure under conditions which permit entry of the molecule into the cell.

Accordingly, the molecules disclosed herein, especially the nucleic acid molecules described herein, may be for introduction into cells.

In another teaching, the disclosure provides a method of degrading one or more target proteins in a population (two or more (a plurality)) of cells. In this way, one or more target proteins can be degraded in a number of cells at once.

There are many ways in which a molecule of the type described herein may be introduced into a cell. These include, for example, techniques which render cells permissive to exogenous compounds. For example, a cell to be introduced a molecule of this disclosure could be rendered permissive by heat shock, sonication and/or electroporation.

Another mechanism that may be exploited may involve the production of a molecule in which the target protein binding moiety has an affinity for an extracellular or cell surface protein. That cell surface protein may be one which when bound by a target protein binding moiety of the disclosed molecule, is internalised within the cell. For example, the target protein binding part of the disclosed molecules may be bi-specific with affinity (or an ability to bind) both a cell surface protein and some other target within a cell. In this way the molecule of this disclosure (for example a fusion construct between an E3 ligase component/RING domain of a SUMO-target E3 ligase and a bi-specific target binding moiety (a bi-specific nanobody for example)) could be target to a specific cell surface protein and also to another intracellular target. Molecules of this type may have significant therapeutic applications as the bi-specific nature of the target protein binding moiety may allow the molecule to be targeted to specific cell types. For example, the molecules could be restricted to a subset of cells which express a particular protein or antigen. After binding the cell surface protein, these molecules would be internalised and would then be able to target an intracellular protein for degradation. Molecules of this type would have particular application in the treatment of cancer when the target protein binding part of the molecule binds a cancer cell marker and an intracellular protein associated with (or causative of) the cancer. Therefore, this disclosure provides a molecule comprising
(a) an E3 ligase component and a bi-specific target protein binding moiety; or
(b) the RING domain of a SUMO-targeted ubiquitin ligase and a bi-specific target protein binding moiety; or
(c) a sequence comprising any one of SEQ ID NOS: 1-270, or a RING domain fragment thereof and a bi-specific target protein binding moiety; or
(d) a sequence comprising any one of SEQ ID NOS: 28-29, or a U-box domain fragment thereof and bi-specific target protein binding moiety.

In each of cases (a)-(d) above, the bi-specific target binding moiety may be a bi-specific antibody or bi-specific nanobody.

In each of cases (a)-(d) above, the bi-specific target binding moiety may exhibit binding specificity or affinity for, for example, an extracellular protein and an intracellular protein,

In each of cases (a)-(d) above, the bi-specific target binding moiety may exhibit binding specificity or affinity for, for example, a cell surface protein an intracellular protein.

The cell surface protein may be one which when bound is internalised (thus internalising the bound molecule of this disclosure).

The cell surface protein may be associated with a disease—for example it may be a disease bio-marker. The cell surface protein may be a CD marker. The cell surface protein may be a SIGLEC molecule. The cell surface protein may be a cancer (or tumour) antigen.

The intracellular protein may be associated with a disease. For example, it may be causative of and/or associated with a disease. The intracellular protein may be associated with a cancer.

A method of degrading an intracellular protein may comprise a step in which the cell (containing the protein to be degraded) is electroporated with a molecule or this disclosure and/or a sequence encoding the same.

The method may additionally comprise the step of inducing expression of a molecule of this disclosure and/or a sequence encoding the same.

As described above, a sequence encoding a molecule of this disclosure may be provided in the form of a vector, for example a plasmid.

The molecules of this disclosure may be used to degrade disease causing proteins. Accordingly, one of skill will appreciated that the targeted degradation of certain proteins may have considerable therapeutic benefit—particularly where the expression of that protein results in some form of disease or condition.

As such, this disclosure also provides the molecules described herein for use in medicine.

The disclosure also provides the molecules of this disclosure for use as medicaments.

Accordingly, this disclosure provides a molecule comprising: (i) an E3 ligase component and a target protein binding moiety; or (ii) the RING domain of a SUMO-targeted ubiquitin ligase and a target protein binding moiety, for use in medicine or for use as a medicament.

In view of the above, the disclosure provides a molecule comprising:
(a) an E3 ligase component and a target protein binding moiety; or
(b) the RING domain of a SUMO-targeted ubiquitin ligase and
a target protein binding moiety; or
(c) a sequence comprising any one of SEQ ID NOS: 1-27, or a RING domain fragment thereof and
a target protein binding moiety; or
(d) a sequence comprising any one of SEQ ID NOS: 28-29, or a U-box domain fragment thereof and
a target protein binding moiety;
for use in medicine or for use as a medicament.

One of skill will readily appreciate that the molecules of this disclosure, which molecules can be used to affected the targeted degradation and/or destruction of one or more specific proteins, can be used in the treatment and/or prevention of any diseases in which the expression of one or more proteins (for example, one or more mutated proteins) is associated with and/or causative of, a disease or condition.

Accordingly, the disclosure provides a molecule comprising: (i) an E3 ligase component and a target protein binding moiety; or (ii) the RING domain of a SUMO-targeted ubiquitin ligase and a target protein binding moiety, for use in the treatment and/or prevention of any disease or condition associated with the expression, for example aberrant expression of a protein (including mutated proteins).

A disease or condition associated with the expression of a protein may be characterised by the over expression of a particular protein, the aberrant expression of one or more proteins and/or the expression of a mutated form of a protein. The molecules of this disclosure may be used to degrade proteins which are mutated, aberrantly or over expressed,

The disclosure provides a molecule comprising:
(a) an E3 ligase component and a target protein binding moiety; or
(b) the RING domain of a SUMO-targeted ubiquitin ligase and
a target protein binding moiety; or
(c) a sequence comprising any one of SEQ ID NOS: 1-27, or a RING domain fragment thereof and
a target protein binding moiety; or
(d) a sequence comprising any one of SEQ ID NOS: 28-29, or a U-box domain fragment thereof and
a target protein binding moiety;

for use in the treatment and/or prevention of any disease or condition associated with the expression, for example aberrant expression of a protein (including mutated proteins).

The molecules of this disclosure may be useful in the treatment or prevention of cell proliferation or cell differentiation disorders. For example, the molecules of this disclosure may be useful in the treatment and/or prevention of cancer. Accordingly, the disclosure provides a molecule comprising:
(a) an E3 ligase component and a target protein binding moiety; or
(b) the RING domain of a SUMO-targeted ubiquitin ligase and
a target protein binding moiety; or
(c) a sequence comprising any one of SEQ ID NOS: 1-27, or a RING domain fragment thereof and
a target protein binding moiety; or
(d) a sequence comprising any one of SEQ ID NOS: 28-29, or a U-box domain fragment thereof and
a target protein binding moiety;

for use in the treatment and/or prevention of a cell proliferation disorder and/or a cell differentiation disorder and/or cancer.

A molecule for use in the treatment of cancer may comprise a target binding moiety with affinity and/or specificity for a protein associated with, or causative of, a cancer.

By way of non-limiting example and without wishing to be bound by theory, a molecule of this disclosure may be exploited (or used) in the treatment and/or prevention of cancer by the targeted degradation of some dominant oncogene. A molecule for this use may comprise (i) an E3 ligase component and a target protein binding moiety; or (ii) the RING domain of a SUMO-targeted ubiquitin ligase and a target protein binding moiety. The target protein binding moiety may in each case have with affinity/specificity for (or which binds to or recognises) a dominant oncogene and not the wild type protein. The target protein binding moiety may be a nanobody.

A Molecule for use in medicine and/or for use in the treatment and/or prevention of one or more types of cancer may comprise (i) an E3 ligase component and a target protein binding moiety; or (ii) the RING domain of a SUMO-targeted ubiquitin ligase and a target protein binding moiety. In each case the target protein binding moiety may target (i.e. bind to, associate with and/or have specificity or affinity for) a protein which is known to be associated with certain cancers. By way of example, a molecule of this disclosure may bind to or have affinity/specificity for the mutated form of Ras and the mutated for of BRAF (mutated in melanoma).

Accordingly, the disclosure provides a molecule comprising: (i) an E3 ligase component and a moiety which binds to Ras; or (ii) the RING domain of a SUMO-targeted ubiquitin ligase and a moiety which binds to Ras. The molecule may comprise a fusion between an E3 ligase component and a moiety which binds Ras or a fusion between a SUMO-targeted ubiquitin ligase and a moiety which binds Ras.

The disclosure also provides a molecule comprising: (i) an E3 ligase component and a moiety which binds to Ras; or (ii) the RING domain of a SUMO-targeted ubiquitin ligase and a moiety which binds to Ras; for use in the treatment of cancer.

In one embodiment, the moiety binds to a mutated form of Ras which is (or is known to be) associated with a cancer. A mutated form of Ras may contain, relative to a wild-type Ras sequence, one or more amino acid substitutions, additions, deletions and/or inversions. A molecule for use in the treatment of cancer may comprise a fusion between an E3 ligase component and a moiety which binds to (a mutated form of) Ras; or a fusion between the RING domain of a SUMO-targeted ubiquitin ligase and a moiety which binds to (a mutated form of) Ras.

The disclosure also provides a molecule comprising: (i) an E3 ligase component and a moiety which binds to BRAF; or (ii) the RING domain of a SUMO-targeted ubiquitin ligase and a moiety which binds to BRAF. The molecule may comprise a fusion between an E3 ligase component and a moiety which binds to BRAF or a fusion between a SUMO-targeted ubiquitin ligase and a moiety which binds to BRAF.

Also disclosed is a molecule comprising: (i) an E3 ligase component and a moiety which binds to BRAF; or (ii) the RING domain of a SUMO-targeted ubiquitin ligase and a moiety which binds to BRAF; for use in the treatment of melanoma.

In one embodiment, the moiety binds to a mutated form of BRAF which is (or is known to be) associated with melanoma. A mutated form of BRAF may contain, relative to a wild-type BRAF sequence, one or more amino acid substitutions, additions, deletions and/or inversions. A molecule for use in the treatment of melanoma may comprise a fusion between an E3 ligase component and a moiety which binds to BRAF; or a fusion between the RING domain of a SUMO-targeted ubiquitin ligase and a moiety which binds to (a mutated form of) BRAF.

A molecule of this disclosure may also be used to degrade the aberrant fusion proteins that can arise in certain cancers. Without wishing to be bound by theory or example, the BCR-ABL fusion occurs in chronic myeloid leukaemia. Therefore a molecule comprising (i) an E3 ligase component and a moiety with affinity or specificity for an aberrant fusion; or (ii) the RING domain of a SUMO-targeted ubiquitin ligase and a moiety with affinity or specificity for an aberrant fusion may be useful in the treatment of these diseases.

Accordingly, the disclosure provides a molecule comprising: (i) an E3 ligase component and a moiety which binds a BCR-ABL fusion; or (ii) the RING domain of a SUMO-targeted ubiquitin ligase and a moiety which binds a BCR-ABL fusion. The molecule may comprise a fusion between an E3 ligase component and a moiety which binds a BCR-ABL or a fusion between a SUMO-targeted ubiquitin ligase and a moiety which binds a BCR-ABL.

The disclosure also provides a molecule comprising: (i) an E3 ligase component and a moiety which binds a BCR-ABL fusion or (ii) the RING domain of a SUMO-targeted ubiquitin ligase and a moiety which binds a BCR-ABL fusion; for use in the treatment of chronic myeloid leukaemia.

A molecule for use in the treatment of chronic myeloid leukaemia may comprise a fusion between an E3 ligase component and a moiety which binds a BCR-ABL or a fusion between a RING domain of a SUMO-targeted ubiquitin ligase and a moiety which binds to a BCR-ABL fusion.

In another (non-limiting) example, the PML-RAR fusion is known to occur in instances of Acute Promyelocytic Leukaemia. As such, and without wishing to be limited to this example, the disclosure provides a molecule comprising (i) an E3 ligase component and a moiety which binds a PML-RAR fusion; or (ii) the RING domain of a SUMO-targeted ubiquitin ligase and a moiety which binds a PML-RAR fusion. The molecule may comprise a fusion between an E3 ligase component and a moiety which binds a PML-RAR fusion or a fusion between a SUMO-targeted ubiquitin ligase and a moiety which binds a PML-RAR fusion.

Also disclosed is a molecule comprising (i) an E3 ligase component and a moiety which binds a PML-RAR fusion; or (ii) RING domain of a SUMO-targeted ubiquitin ligase and a moiety which binds a PML-RAR fusion; for use in the treatment of Acute Promyelocytic Leukaemia.

A molecule for use in the treatment of Acute Promyelocytic Leukaemia may comprise a fusion between an E3 ligase component and a moiety which binds a PML-RAR fusion or a fusion between the RING domain of a SUMO-targeted ubiquitin ligase and a moiety which binds to a PML-RAR fusion.

As stated, the over-expression of certain proteins is also known to be associated with disease, including cancer. A molecule of this disclosure (with its ability to target and degrade specific proteins) could be used to treat disease by degradation of any over-expressed proteins. For example, in the case of Burkitt's Lymphoma (and without wishing to be bound by theory) Myc is over expressed. Thus a molecule comprising (i) an E3 ligase component and a moiety which specifically binds Myc; or (ii) the RING domain of a SUMO-targeted ubiquitin ligase and a moiety which specifically binds Myc, may be used to degrade at least a portion of the over-expressed Myc. The administration of any such protein may require control over the dose used so that the total amount of protein degradation is controlled—this is necessary as normal cell function may require some level of expression of the protein to be degraded.

Accordingly, this disclosure provides a molecule comprising: (i) an E3 ligase component and a moiety which specifically binds Myc; or (ii) the RING domain of a SUMO-targeted ubiquitin ligase and a moiety which binds Myc. The molecule may comprise a fusion between an E3 ligase component and a moiety which specifically binds Myc or a fusion between a SUMO-targeted ubiquitin ligase and a moiety which Myc.

There is also provided a molecule of this disclosure for use in the treatment and/or prevention of Burkitt's Lymphoma. For example, the disclosure provides a molecule comprising: (i) an E3 ligase component and a moiety which specifically binds Myc; or (ii) the RING domain of a SUMO-targeted ubiquitin ligase and a moiety which binds Myc; for use in the treatment or prevention of Burkitt's Lymphoma.

In a number of neurodegenerative diseases, mutant or misfolded protein accumulates. Examples include Prion diseases such as mad cow disease, CJD or scrapie. A molecule of this disclosure, with its ability to target specific proteins can be used to treat or prevent neurodegenerative diseases. In such cases, and without wishing to be bound by theory, the target protein binding moiety part of the disclosed molecule (for example the nanobody part) may be used to preferentially bind the misfolded protein (rather than any wild-type or correctly folded protein)—this would lead to the selective degradation of any misfolded protein.

Thus, this disclosure provides a molecule comprising: (i) an E3 ligase component and a moiety which binds a misfolded protein; or (ii) the RING domain of a SUMO-targeted ubiquitin ligase and a moiety which binds a misfolded protein. In one embodiment, the mi-folded protein is associated with, or causative of, a neurodegenerative disease, disorder or condition.

The molecule may comprise a fusion between an E3 ligase component and a moiety which binds a misfolded protein or a fusion between a SUMO-targeted ubiquitin ligase and a moiety which binds a mis-folded protein.

It should be noted that the term “mis-folded” protein relates to any protein which exhibits a folding patter, confirmation or organisation, which is different to the folding pattern, confirmation or organisation of a wild-type protein of the same type. For example, a “mis-folded protein” may have a different tertiary/quaternary sequence to the tertiary/quaternary sequence of the corresponding wild-type protein.

Also disclosed is a molecule comprising (i) an E3 ligase component and a moiety which binds a misfolded protein; or (ii) the RING domain of a SUMO-targeted ubiquitin ligase and a moiety which binds a misfolded protein; for use in the treatment or prevention of a neurodegenerative disease, wherein the misfolded protein is associated with, or causative of, the neurodegenerative disease. Without wishing to be bound by any particular theory or use, the molecules described herein may be for use in the treatment of diseases such as Alzheimer's disease. Alzheimer's disease may be associated with the development of amyloid plaques within a cell. The molecules described herein may be used to affect the targeted degradation of those amyloid plaques. A molecule for use in the treatment or prevention of diseases such as Alzheimer's disease may comprise the RING domain of a SUMO-targeted ubiquitin ligase and moiety capable of binding an amyloid plaque.

Other diseases may be associated with an accumulation of poly glutamine (PolyQ) and a molecule of this disclosure may be used to treat or prevent a disease or condition associated with, or characterised b,y PolyQ accumulation and/or PolyQ tract formation. Such diseases may include, for example, those diseases referred to as PolyQ expansion diseases, spinal bulbar muscular atrophy, dentatorubral pallidoluysian atrophy, Huntington's disease (HD), and spinocerebellar ataxia.

Thus a molecule of this disclosure may be used to treat or prevent PolyQ expansion diseases, spinal bulbar muscular atrophy, dentatorubral pallidoluysian atrophy, Huntington's disease (HD), and spinocerebellar ataxia.

Accordingly, the disclosure provides a molecule comprising: (i) an E3 ligase component and a moiety which binds PolyQ and/or a PolyQ tract; or (ii) the RING domain of a SUMO-targeted ubiquitin ligase and a moiety which binds PolyQ and/or a PolyQ tract. The molecule may comprise a fusion between an E3 ligase component and a moiety which binds PolyQ and/or a PolyQ tract or a fusion between a SUMO-targeted ubiquitin ligase and a moiety which binds PolyQ and/or a PolyQ tract.

Also disclosed is a molecule comprising: (i) an E3 ligase component and a moiety which binds PolyQ and/or a PolyQ tract; or (ii) the RING domain of a SUMO-targeted ubiquitin ligase and a moiety which binds PolyQ and/or a PolyQ tract; for use in the treatment or prevention of one or more diseases selected from the group consisting of:
(i) PolyQ expansion diseases;
(ii) spinal bulbar muscular atrophy;
(iii) dentatorubral pallidoluysian atrophy;
(iv) Huntington's disease (HD); and
(v) spinocerebellar ataxia

A further application of the molecules describe herein is in genetic engineering. For example, a molecule of this disclosure could be designed to bind to and degrade a protein which is somehow essential to the pathogenicity, life cycle and/or replication of a particular pathogen.

A molecule of this type could be introduced into a cell—that cell may then become resistant to that pathogen as upon infection (or entry into the cell) the molecule would target the degradation of the pathogen (essential) protein—this would neutralise, kill and/or inhibit the pathogen.

The pathogen (to which the protein is essential) may be an intracellular pathogen or a bacterial, viral or fungal pathogen.

Accordingly, the disclosure provides a molecule comprising: (i) an E3 ligase component and a moiety which binds a pathogen essential protein; or (ii) the RING domain of a SUMO-targeted ubiquitin ligase and a moiety which binds a pathogen essential protein. The molecule may comprise a fusion between an E3 ligase component and a moiety which binds a pathogen essential protein or a fusion between a SUMO-targeted ubiquitin ligase and a moiety which binds a pathogen essential protein.

One of skill will appreciate that a molecule of this type may be used to render a cell resistant to a pathogen. In other words, the molecule may alter the response of the cell to the pathogen.

In one embodiment, a molecule of this disclosure may be used to render a cell resistant to a virus infection. For example a molecule of this disclosure may be used to render a cell resistant to an HIV infection. Without wishing to be bound by theory, an individual's T-cells (or a population or quantity thereof) could be removed and engineered to express a molecule of this disclosure comprising a moiety which binds an essential HIV protein. In such cases, a cell which comprises (or expresses) a molecule of this disclosure, which molecule comprises a moiety which binds an essential HIV protein, is able to defeat the infection as when the virus enters the cell, the molecule is able to direct the targeted degradation of the essential HIV protein. This would neutralise, destroy or inactivate the HIV particle. A subject may be repopulated with these transformed cells which would expand and be individually resistant to HIV infection.

Thus, disclosed herein is a molecule comprising: (i) an E3 ligase component and a moiety which binds an essential HIV protein; or (ii) the RING domain of a SUMO-targeted ubiquitin ligase and a moiety which binds an essential HIV protein. The molecule may comprise a fusion between an E3 ligase component and a moiety which binds an essential HIV protein or a fusion between a SUMO-targeted ubiquitin ligase and a moiety which binds an essential HIV protein.

Also disclosed is a molecule comprising the (i) an E3 ligase component and a moiety which binds an essential HIV protein; or (ii) RING domain of a SUMO-targeted ubiquitin ligase and a moiety which binds an essential HIV protein, for use in the treatment and/or prevention of an HIV infection. As stated, the molecule for use may be a fusion between an E3 ligase component and a moiety which binds an essential HIV protein or a fusion between a SUMO-targeted ubiquitin ligase and a moiety which binds an essential HIV protein.

The molecules of this disclosure may be used to treat or prevent viral, fungal and/or bacterial infections, wherein the molecules of this disclosure comprise the subset of molecules which comprise wither an E3 ligase component or a RING domain of a SUMO-targeted binding ubiquitin ligase and a moiety which binds an essential viral, bacterial and/or fungal protein.

Accordingly, the disclosure provides a molecule comprising (i) an E3 ligase component and a moiety which binds an essential viral, bacterial and/or fungal protein; or (ii) RING domain of a SUMO-targeted ubiquitin ligase and a moiety which binds an essential viral, bacterial and/or fungal protein. The molecule may comprise a fusion between an E3 ligase component and a moiety which binds an essential viral, bacterial and/or fungal protein or a fusion between a SUMO-targeted ubiquitin ligase and a moiety which binds an essential viral, bacterial and/or fungal protein.

Also disclosed is (i) an E3 ligase component and a moiety which binds an essential viral, bacterial and/or fungal protein; or (ii) the RING domain of a SUMO-targeted ubiquitin ligase and a moiety which binds an essential viral, bacterial and/or fungal protein for use in the treatment and/or prevention of a viral, bacterial and/or fungal infection.

The term “cell” as used herein may include, for example, any animal, mammalian, insect and/or plant cell—any of which can be modified to express a molecule of this disclosure.

In view of the above, the disclosure provides:
(i) a modified cell
(ii) a modified animal cell
(iii) a modified mammalian cell; or
(iv) a modified plant cell; or
(v) a modified insect cell;

wherein the modified cell is modified to express a molecule of this disclosure or a molecule comprising:
(i) an E3 ligase component and a moiety which binds a target protein; or
(ii) the RING domain of a SUMO-targeted ubiquitin ligase and
a moiety which binds a target protein.

In one embodiment the modified cell is modified to express a molecule comprising:
(i) an E3 ligase component and a moiety which binds an essential viral, bacterial and/or fungal protein; or
(ii) the RING domain of a SUMO-targeted ubiquitin ligase and
a moiety which binds an essential viral, bacterial and/or fungal protein.

Moreover, this disclosure provides a modified plant cell or a modified insect cell or a modified mammalian cell (modified as described above to express a molecule of this disclosure) for use in the treatment of a viral, fungal or bacterial infection.

A method of treating a viral, fungal and/or bacterial infection may include, for example, the following steps:
(i) introducing into a cell, a molecule comprising
(a) an E3 ligase component and a moiety which binds a target protein (for example an essential viral, bacterial and/or fungal protein); or
(b) a RING domain of a SUMO-targeted ubiquitin ligase and a moiety which binds a target protein (for example an essential viral, bacterial and/or fungal protein); and
(ii) inducing the expression of the molecule in the cell.

The method may be used to treat any specific disease, condition or infection by providing a molecule which comprises (i) an E3 ligase component and a moiety which binds a protein essential to a pathogen which is associated with the disease, condition or infection to be treated; or (ii) RING domain of a SUMO-targeted ubiquitin ligase and a moiety which binds a protein essential to a pathogen which is associated with the disease, condition or infection to be treated.

As stated, the term “essential” may refer to a protein which is required by the pathogen for successful infection, host cell entry, replication, pathogenesis and/or release.

The disclosure further provides compositions comprising a molecule of this disclosure. The disclosure further provides compositions comprising vectors and cells disclosed herein.

A composition may comprise a molecule, vector or cell as described herein and one or more excipients, diluents or buffers.

The composition may be a pharmaceutical composition.

The composition may comprise one or more pharmaceutically acceptable excipients, diluents and/or buffers.

The composition may be sterile.

The composition may be formulated for oral, parenteral, mucosal, nasal (intranasal) or intravenous administration.

The disclosure further provides methods of making the molecules described herein.

The molecules of this disclosure may be prepared using molecular and/or recombinant techniques.

A method of making a molecule of this disclosure may comprise fusing an E3 ligase component to a target protein binding moiety. This provides an E3 ligase:target protein binding moiety fusion.

A method of making a molecule of this disclosure may comprise fusing a U-box domain to a target protein binding moiety. This provides a U-box domain:target protein binding moiety fusion.

A method of making a molecule of this disclosure may comprise fusing a sequence provided by any one of SEQ ID NOS: 1-29 to a target protein binding moiety.

All fusion constructs as created by the above methods may include a linker moiety (for example a peptide linker) linking the E3 ligase component, the U-box domain or the sequence derived from any of SEQ ID NOS; 1-29, to the target protein binding moiety.

As stated, the target protein binding moiety may be a nanobody with specificity for a protein to be degraded.

A method of making a molecule of this disclosure may comprise modifying the RING domain of a SUMO-targeted ubiquitin ligase to include a target protein binding protein.

A method of making a molecule of this disclosure may comprise modifying the RING domain of a SUMO-targeted ubiquitin ligase to include a nanobody, for example a camelid-derived single-domain nanobody, wherein the nanobody has specificity and/or affinity for a target protein. For example, the SUMO recognition domain of the RING domain of the SUMO-targeted ubiquitin ligase may be replaced with the appropriate target protein binding moiety or the appropriate (camelid) nanobody.

A method of making a molecule of this disclosure may comprise the step of making a RING domain:target protein binding moiety fusion—for example a RING domain:nanobody fusion.

A method of making a molecule of this disclosure may comprise modifying the RING domain of a SUMO-targeted ubiquitin ligase to include a target binding moiety.

For example, the SUMO recognition domain of the RING domain of the SUMO-targeted ubiquitin ligase may be replaced with a target binding moiety. In this way the substrate recognition properties of the RING domain of a SUMO-targeted ubiquitin ligase are altered so that the resulting construct becomes specific (or shows affinity for) the target of the target binding moiety.

The disclosure further provides kits, the kits comprising components selected from the group consisting of:
(a) a molecule of this disclosure;
(b) a nucleic acid encoding a molecule of this disclosure;
(c) a vector comprising a sequence encoding a molecule of this disclosure
(d) a cell able to receive a molecule of this disclosure and/or a vector of this disclosure; and
instructions for use.

The kits may comprise reagents, buffers and other compositions for use in methods of degrading proteins/target proteins.

### DETAILED DESCRIPTION

The present disclosure will now be described by reference to the following figures which show:

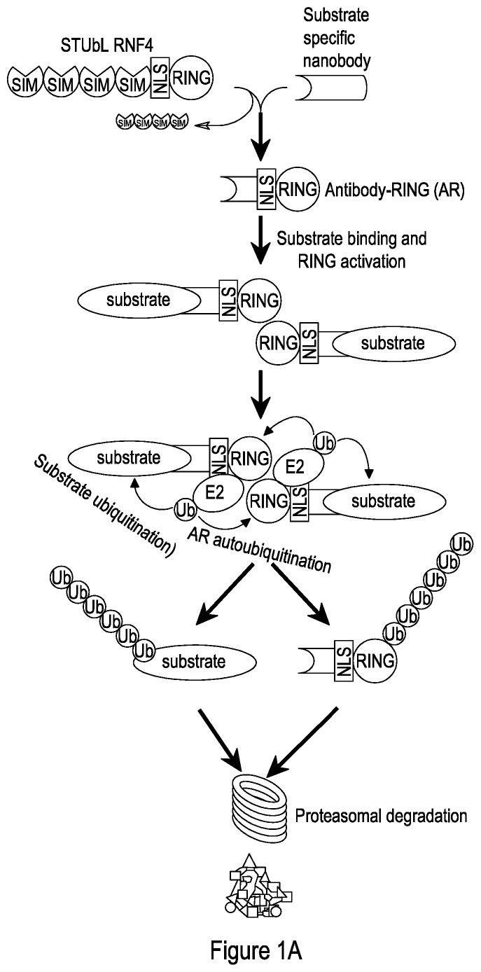

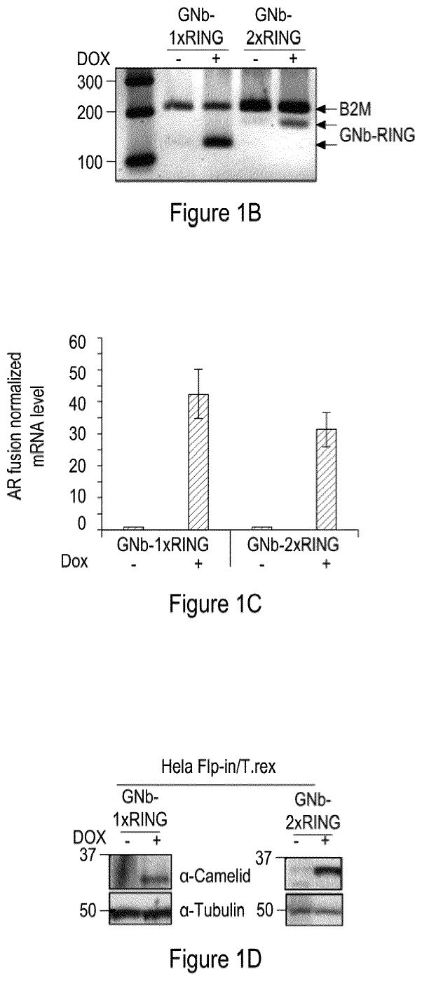

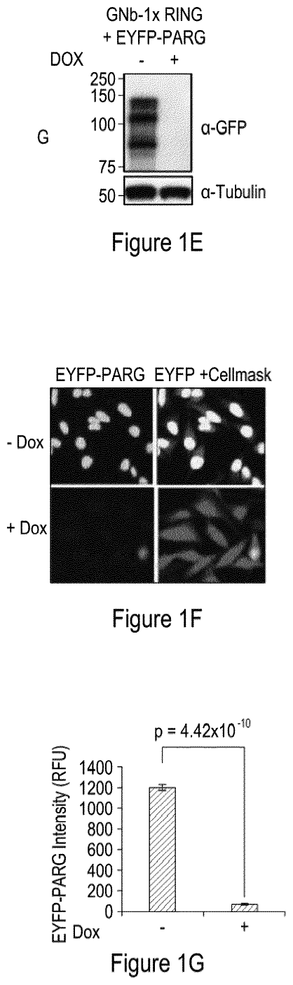

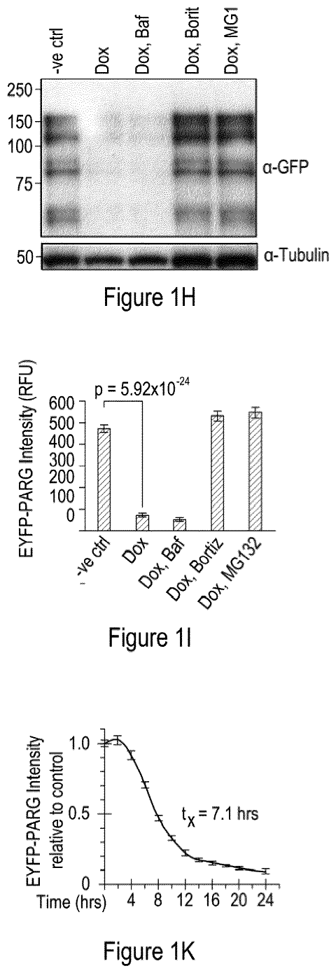

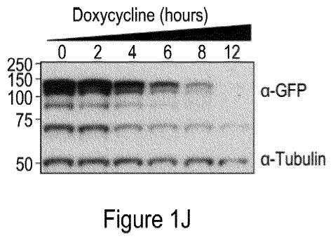

FIG. 1: Antibody-RING Mediated Destruction (ARMeD)-principle, tool development and degradation of EYFP-PARG. (A) Schematic representation of the principle of Antibody-RING Mediated Destruction (ARMeD). The SUMO recognition motifs (SIMs) of the SUMO targeted ubiquitin ligase (STUbL) RNF4 are replaced with a nanobody targeting a protein substrate of interest. Expression of this fusion protein allows binding of substrate to the nanobody and RING mediated ubiquitination leading to proteasomal degradation. Hela Flp-in/T.Rex cells engineered to inducibly express GNb-1xRING or GNb-2xRING were either untreated (−) or Doxycycline treated (+) for 24 hr. mRNA levels were analysed by qRT-PCR with beta-2 microglobulin (B2M) as housekeeping control and the products at 24 cycles were separated on an agarose gel (B). Quantitative expression data were obtained from three independent RNA preparations from each condition, normalized to B2M mRNA and uninduced control samples. Error bars represent mean±SD from three independent replicates (C). Protein levels were analysed by western blotting using an anti-camelid antibody (D). Hela Flp-in/T. Rex cells engineered to inducibly express GNb-1xRING and stably express YFP-PARG were induced with Doxycycline as above and protein levels analysed by western blotting using an anti-GFP antibody (E), or cells were grown in 96-well plates fixed and visualised by high-content (HC) imaging using IN Cell analyser 2000 (F). The HC data were obtained from 152,668 (uninduced) or 80,745 (induced) cells in 6 wells, and quantitication of intracellular YFP was performed using the InCell Developer toolbox. YFP intensity data are plotted as the mean of 6 wells±SD (G). To establish the pathway of protein degradation, cells were incubated with autophagy inhibitor bafilomycin A1 (Baf, 100 nM) or proteasome inhibitors bortizomib (1 μM) or MG132 (10 μg/ml) for 1.5 hr prior to induction with Doxycycline for 16 hr. Western blotting (H) and HC analysis (I) were performed as above. The HC YFP-PARG intensity data were obtained from 20,000-40,000 cells grown in 12 wells of a 96 well plate for each condition and plotted as the mean of the 12 well replicates±SD (I). The rate of YFP-PARG degradation was assessed in a time course experiment by collecting cells at the indicated times after Doxycycline addition. Samples were analysed by western blotting (J), or HC imaging (K). The HC YFP intensity data were obtained from 115,000-250,000 cells grown in 8 wells of a 96 well plate for each time point, normalised to the uninduced control cells, and plotted as the mean of the 8 well replicates±SD. Statistical analysis was performed by a two-tailed unpaired t test.

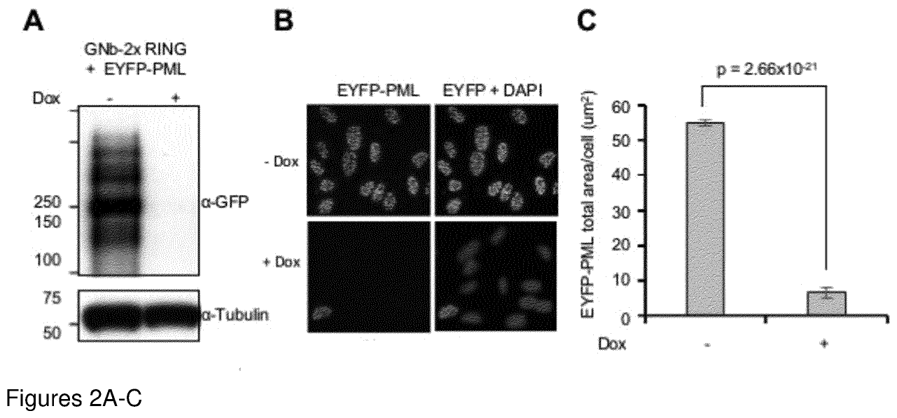

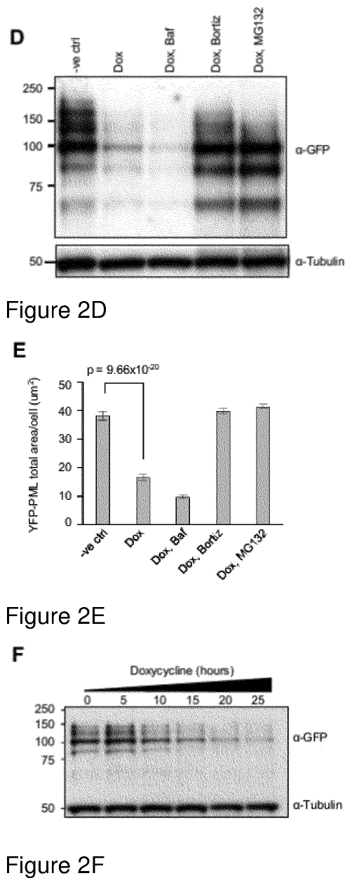

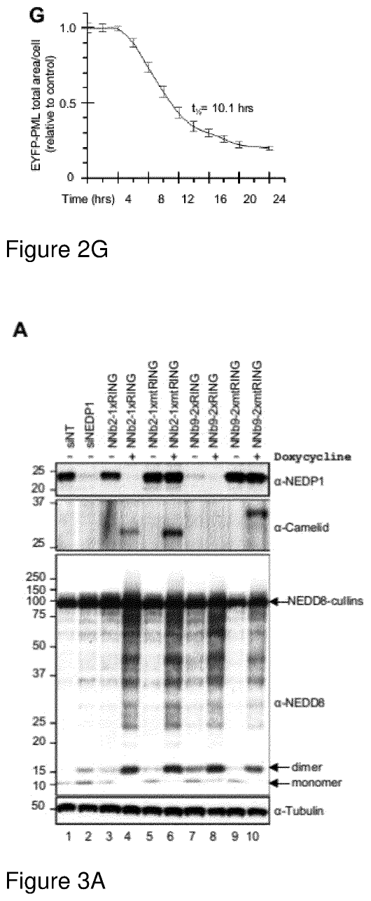

FIG. 2: Antibody-RING Mediated Destruction (ARMeD) of YFP-PML. Hela Flp-in/T.Rex cells engineered to inducibly express GNb-2xRING and stably express YFP-PML were either untreated (−) or Doxycycline treated (+) for 24 hr. Protein levels were analysed by western blotting using an anti-GFP antibody (A), or analysed by high-content (HC) imaging using IN Cell analyser 2000 (B). HC data were obtained from 33,775 (uninduced) or 33,434 (induced) cells in 9 wells, and quantification of YFP fluorescence was performed using the InCell Developer toolbox. Data representing YFP-PML total area/cell are plotted as means of 9 wells±SD (C). To establish the pathway of protein degradation, cells were incubated with autophagy inhibitor bafilomycin A1 (Baf, 100 nM) or proteasome inhibitors bortizomib (1 μM) or MG132 (10 μg/ml) for 1.5 hr prior to induction with Doxycycline for 16 hr. Western blotting (D) and HC analysis (E) were performed as above. The HC YFP-PML data (total area/cell) were obtained from 20,000-50,000 cells grown in 12 wells of a 96 well plate for each condition and plotted as the mean of the 12 well replicates±SD (E). The rate of YFP-PML degradation was assessed in a time course experiment by collecting cells at the indicated times after Doxycycline addition and performing western blotting (F), or high-content analysis (G). The HC EYFP-PML data (total area/cell) were obtained from a total of 20,000-25,000 cells grown in a total of 8 wells of a 96 well plate for each time point, normalised to the uninduced control cells, and plotted as the mean of the 8 well replicates±SD. Statistical analysis was performed by a two-tailed unpaired t test.

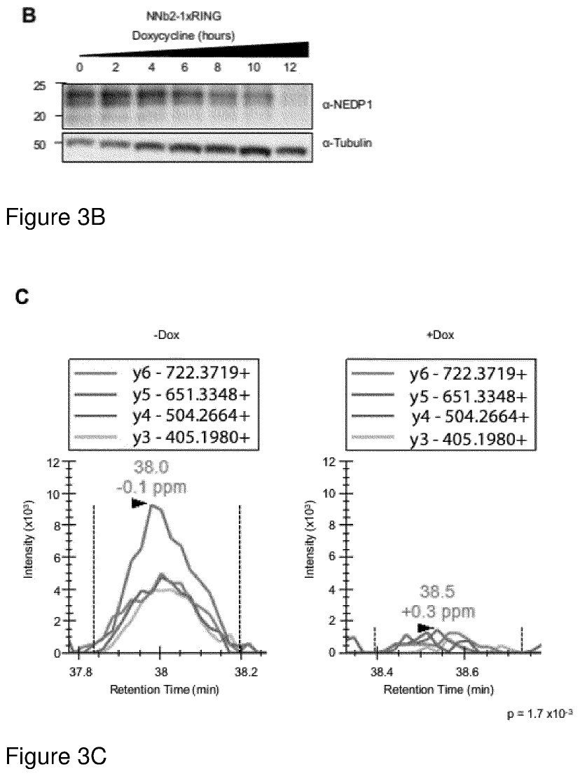

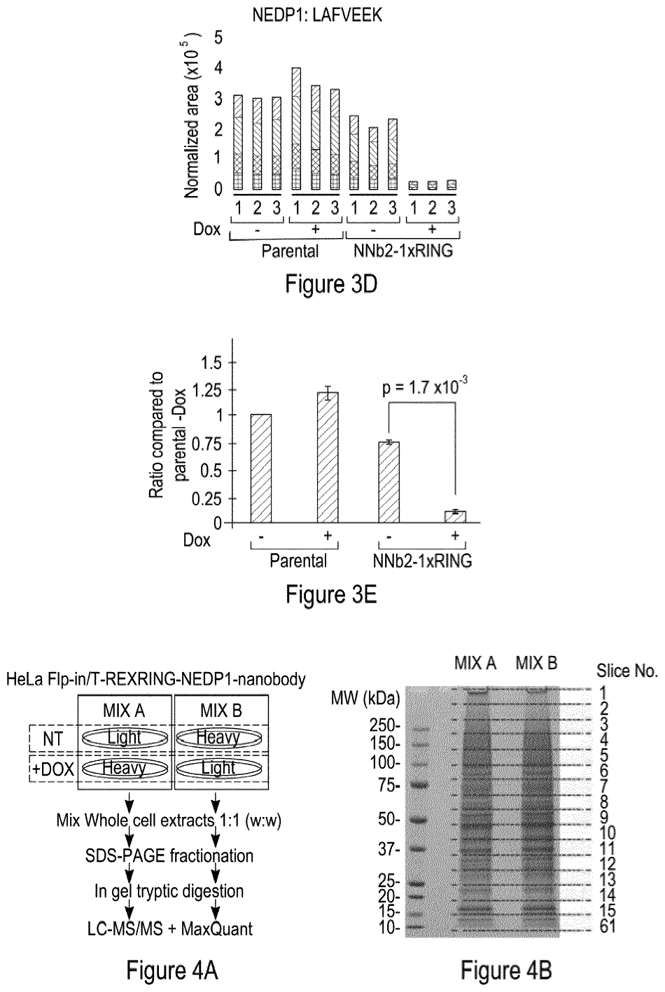

FIG. 3: Degradation of endogenous NEDD8 protease NEDP1. (A) Hela Flp-in/T.Rex cells were transfected with non-targeting (siNT, lane 1) or NEDP1 (siNEDP1, lane2) siRNA, and cell extracts were harvested 72 hours after transfection. Lanes 3-10: Hela Flp-in/T.Rex cells engineered to inducibly express NEDP1 specific nanobody-RING constructs were either untreated (−) or Doxycycline treated (+) for 24 hr. Protein levels were analysed by western blotting using anti-NEDP1, anti-camelid and anti-NEDD8 antibodies, respectively. a-Tubulin was used as loading control. NEDD8-cullins and NEDD8 monomers and dimers are indicated by arrows. (B) The rate of NEDP1 degradation was assessed in a time course experiment by collecting cells at the indicated times after Doxycycline addition and performing western blotting using anti-NEDP1 and anti-Tubulin antibodies. (C) Multiple Reaction Monitoring to quantify NEDP1 depletion. Example MS2 chromatograms for fragment ions y3-y6 of the NEDP1 peptide LAFVEEK with and without doxycycline treatment. Dashed lines are peak boundaries as reported by Skyline. Mass errors for most prominent peaks are labelled as ppm. (D) Example MS2 peak areas used for quantification of NEDP1 ARMeD knockdown (NNb2-1xRING) and control strain (Parental). (E) Median enrichment or depletion compared to PARENTAL-dox of the LAFVEEK, LEAFLGR, and QVAEKLEAFLGR peptides. Error bars are standard error. Statistical analysis was performed by a two-tailed unpaired t test.

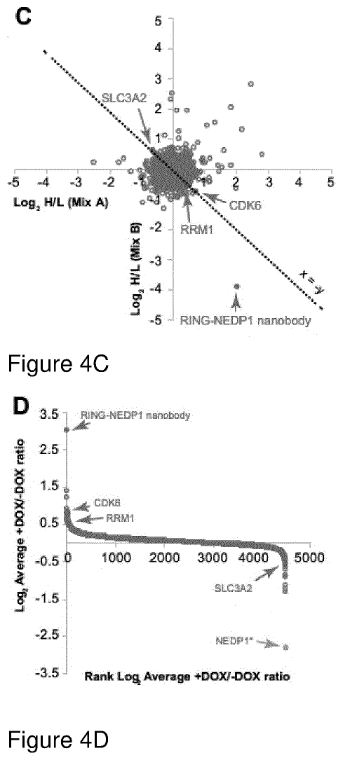

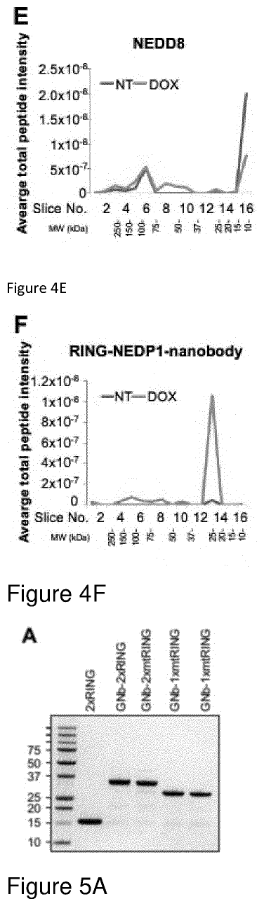

FIG. 4: Total proteome consequences of nanobody-RING fusion expression. (A) Design of a SILAC experiment to identify protein abundance changes to cells after Doxycycline induction of NNb2-1xRING. (B) Gel image of whole cell extracts from SILAC mixes as shown from panel A. (C) Scatter plot showing the SILAC H/L ratio data for the 4506 proteins common to the data derived from the two SILAC mixes. Grey markers indicate proteins not identified as significantly different in both comparisons, nor consistently responding to Doxycycline. Red markers are proteins with significant ratios and consistent response to Doxycycline across both SILAC mixes for the 4506 proteins quantified in all conditions. CDK6—Cyclin dependent kinsae 6, RRM1—Ribonucleoside-diphosphate reductase large subunit, SLC3A2-4F2 cell-surface antigen heavy chain. * Included for comparison; NEDP1 data derived from PRM experiment shown in FIG. 3 and not from this SILAC experiment. (E) Slice-specific total protein intensity data for NEDD8 and the NNb2-1xRING fusion. For each slice, the average intensity across both mixes is shown.

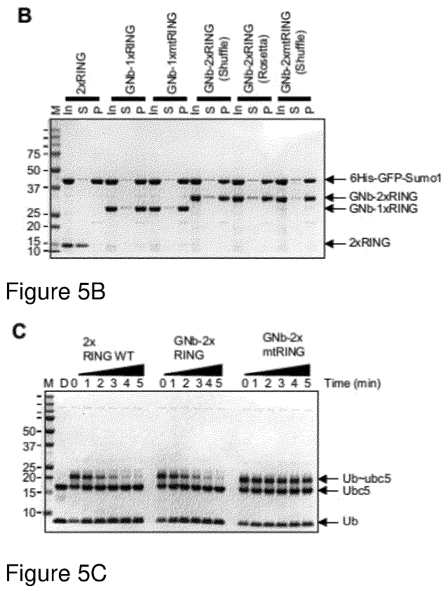

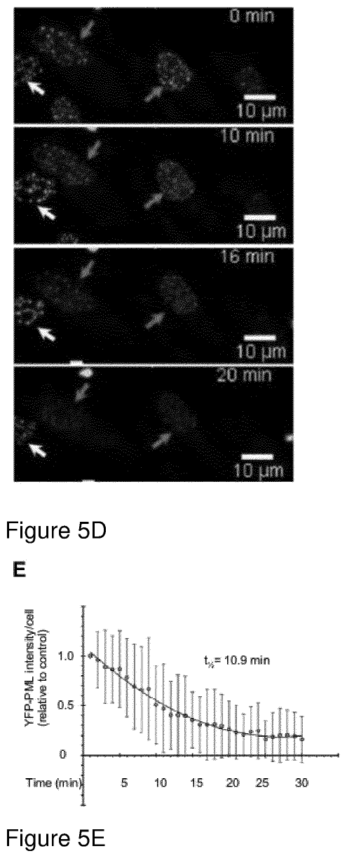

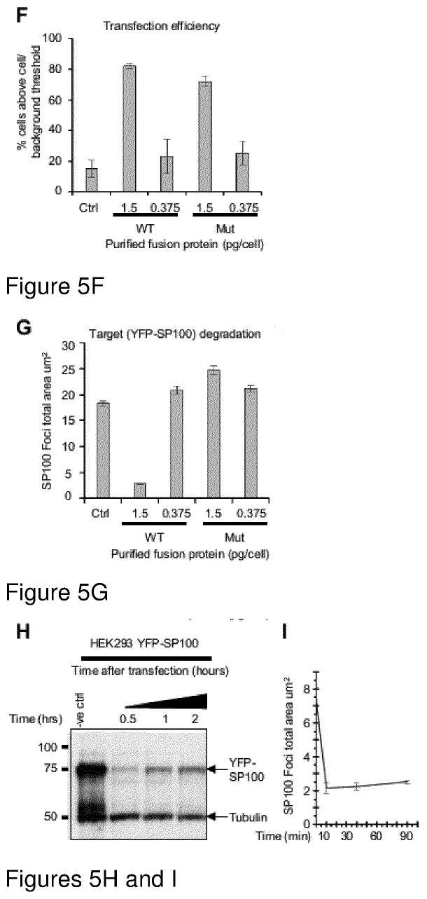

FIG. 5: Acute and rapid degradation of target proteins by ARMeD proteins. (A) Coomassie-blue stained SDS-PAGE analysis of purified GFP nanobody-RING fusions, WT (GNb-1xRING and GNb-2xRING) and (GNb-1xmtRING and GNb-2xmtRING). (B) Nickel bead pull-down assays of recombinant 6His-GFP-SUMO1 with nanobody-RING fusions were evaluated with SDS-PAGE and Coomassie staining (I: input; S: supernatant; P: pulldown), Fused RNF4 RING (2xRING) is used as negative control. (C) Lysine discharge assays with ubiquitin loaded Ubc5 (Ub-Ubc5) in the presence of fused RNF4 RING (2xRING), GNb-2xRING and GNb-2xmtRING. Samples were removed at the indicated times (minutes) and analysed by non-reducing SDS-PAGE. A sample reduced with DTT is indicated. (D) Hela Flp-in/T.Rex cells stably expressing YFP-PMLIII were injected with a 1:1 mixture of GNb-2xRING and mCherry-SIM and images collected every 2 minutes. Injected cells (red arrow) and uninjected cells (yellow arrow) are indicated. The images shown were taken at 0, 10, 16 and 20 min following injection. Injected cells were identified using the mCherry fluorescence and the Mean summed YFP intensity of the injected cells was obtained following background subtraction and plotted (E) for each time point±SD. Efficiency of (F) purified protein delivery to cells and (G) target protein degradation. HEK293 cells stably expressing YFP-SP100 were electroporated with a mix of mCherry-SIM protein and either GNb-2xRING or GNb-2xmtRING (either 0.375 pg or 1.5 μg of each purified protein/cell) and (F) mCherry or (G) YFP fluorescence analysed by high-content (HC) imaging using IN Cell analyser 2000. HC data were obtained from 29923/26007 (0.375/1.5 pg GNb-2xRING/cell) or 21901/32866 (0.375/1.5 pg GNb-2xmtRING/cell) cells in 12 wells, and quantitation of each fluorescence signal was determined individually using the InCell Developer toolbox. Data representing the percentage of cells with mCherry fluorescence above background are plotted as means of 12 wells±SD (F); and YFP fluorescence representing PML/SP100 foci total area/cell are plotted as means of 12 wells±SD (G). (H-I) The rate of NEDP1 degradation was assessed in a time course experiment by electroporating the HEK293 YFP-SP100 cells above with 12 μg of purified GNb-2xRING in a total volume of 100 μl and collecting samples at the indicated times after electroporation and performing western blotting, using an anti-GFP antibody and an anti-tubulin antibody as loading control (H), or HC imaging (I). The total number of analysed cells was 12719 (control), 7745 (10 min), 8480 (40 min) and 14983 (90 min) anthe plotted values represent the SP-100 foci total area/cell averaged from four wells±SD. Statistical analysis was performed by a two-tailed unpaired t test.

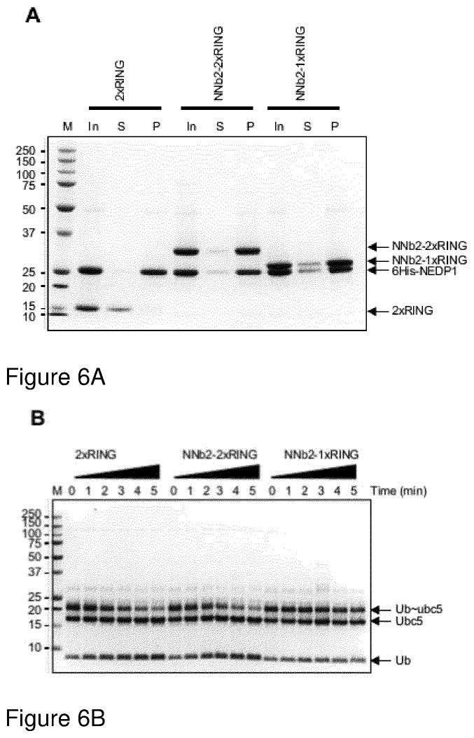

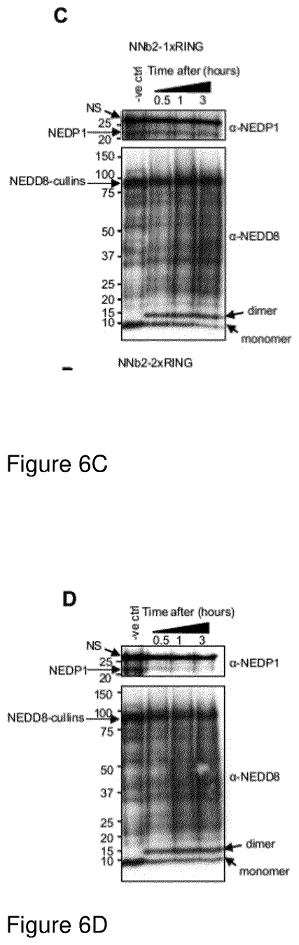

FIG. 6: Rapid Antibody—RING—mediated destruction of endogenous NEDP1. (A) Nickel bead pull-down assays of recombinant 6His-NEDP1 with nanobody-RING fusions (NNb2-1xRING, NNb2-2xRING) were evaluated with SDS-PAGE and Coomassie staining (In: input; S: supernatant; P: pulldown), Fused RNF4 RING (2xRING) is used as negative control. (B) Lysine discharge assays with ubiquitin loaded Ubc5 (Ub-Ubc5) in the presence of fused RNF4 RING (2xRING), NNb2-1xRING and NNb2-2xRING. Samples were removed at the indicated times (minutes) and analysed by non-reducing SDS-PAGE. (C-D) HEK293 cells were electroporated with NNb-1xRING (C) or NNb-2xRING (D) and harvested at the indicated time point after electroporation. Whole cell extracts were separated by SDS-PAGE and analysed by western blotting using NEDP1 and NEDD8 antibodies as indicated. NEDP1, a non-specific band (NS), NEDD8-cullins and NEDD8 monomers and dimers are indicated by arrows.

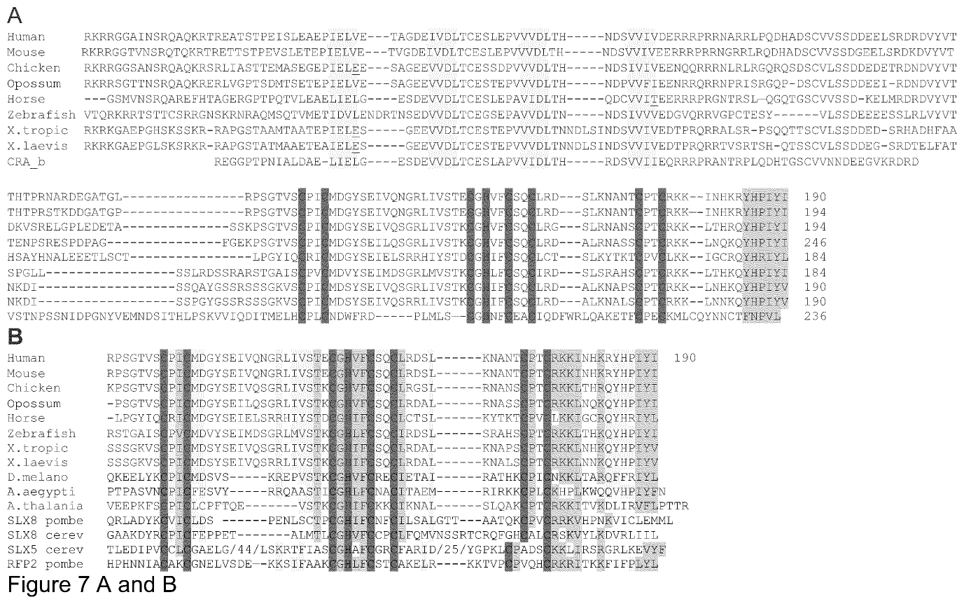

FIG. 7: (A) Sequence pileup of RNF4 proteins. The 7 cysteines and 1 histidine that co-ordinate Zinc in the RING domain are in red. The SUMO interaction motifs (SIMS) the constitute the substrate binding domain are in yellow. A region allowing the RING domain to dimerise is in green. (B) RNF4 RING domains from diverse species.

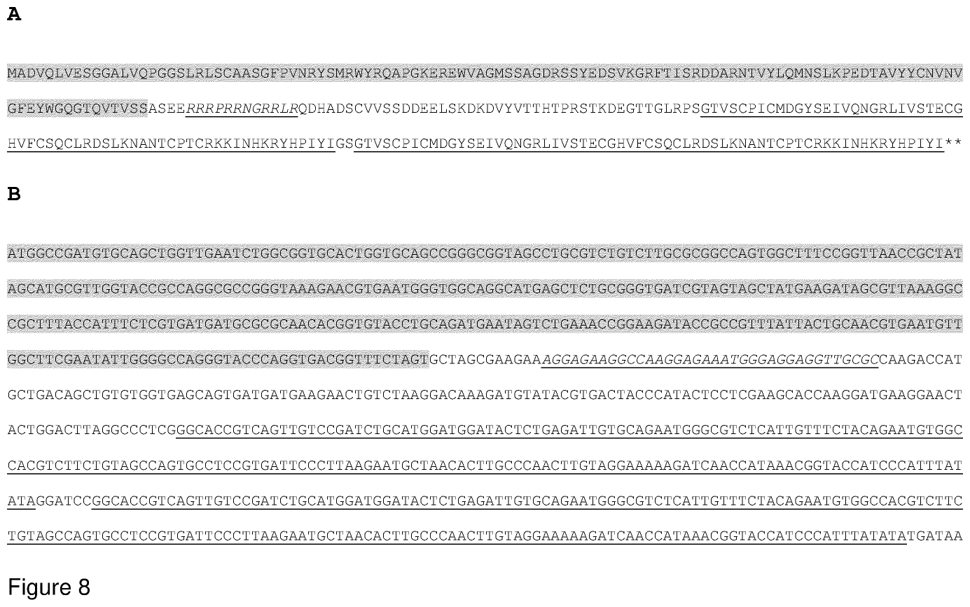

FIG. 8: (A) GNb-2xRING: protein sequence of a fusion protein comprising a nanobody component fused to the RNF nuclear localisation signal and 2×RNF4 RING domain sequences (SEQ ID NO: 30): (B) GNb-2xRING CDS: nucleic acid sequence of the same molecule—encoding a fusion comprising a nanobody, a RNF nuclear localisation domain and 2×RNF4 RING domains. In both cases, the nanobody has specificity for GFP (SEQ ID NO: 31). Key to sequences: grey highlight=target protein binding moiety (nanobody) sequence; italic underlined=nuclear localisation sequence (NLS: in this example this is the RNF NLS); underlined sequence=E3 ligase component (in this example this is the RNF4 RING domain sequence).

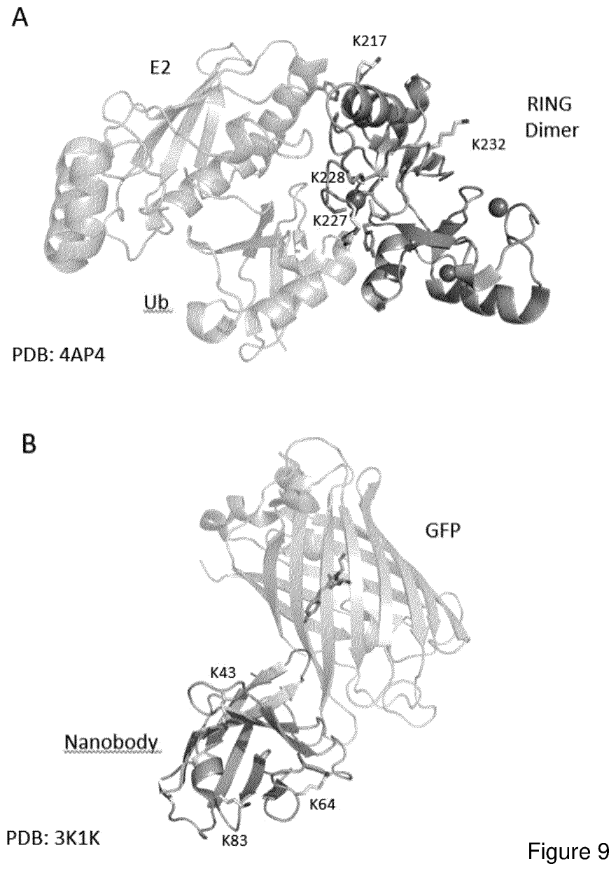

FIG. 9: Location of lysine residues in RING and nanobody. A. structure of the RNF4 RING dimer bound to ubiquitin loaded E2 (PDB: 4AP4) showing selected lysine residues (yellow). Other lysine residues not visible in this view. None of the lysine residues are predicted to interact with the ubiquitin loaded E2. B. Structure of complex between GFP nanobody and GFP (PDB: 3K1K) with lysine residues indicated (yellow). None of the lysine residues are predicted to interact with GFP.

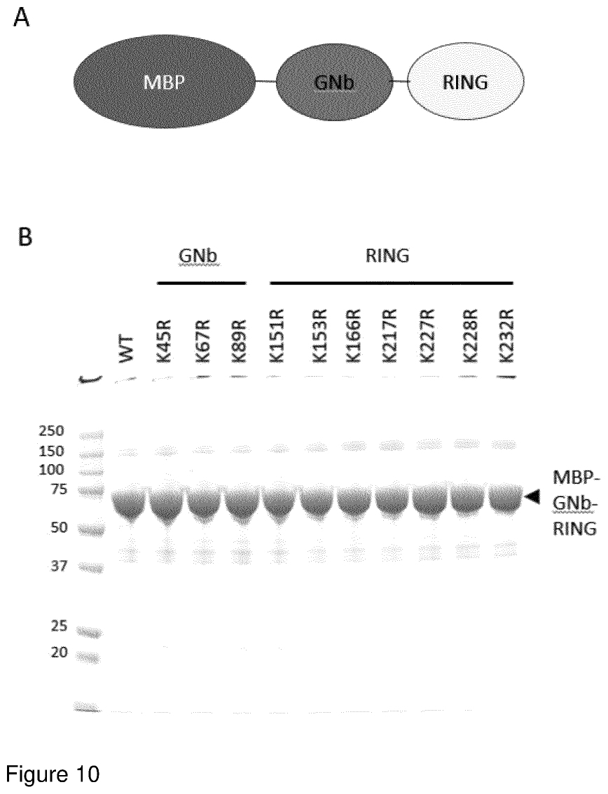

FIG. 10: Purification of MBP-GNb-RING lysine to arginine mutants expressed in bacteria. A. Cartoon of the MBP-GNb-RING fusion. B. WT and mutant versions of MBP-GNb-RING were expressed in bacteria and the proteins applied to Amylose Sepharose resin. After extensive washing bound protein was eluted with 20 mM maltose and 20 microgram of eluted protein analysed by SDS polyacrylamide gel electrophoresis and staining with Coomassie Brilliant Blue.

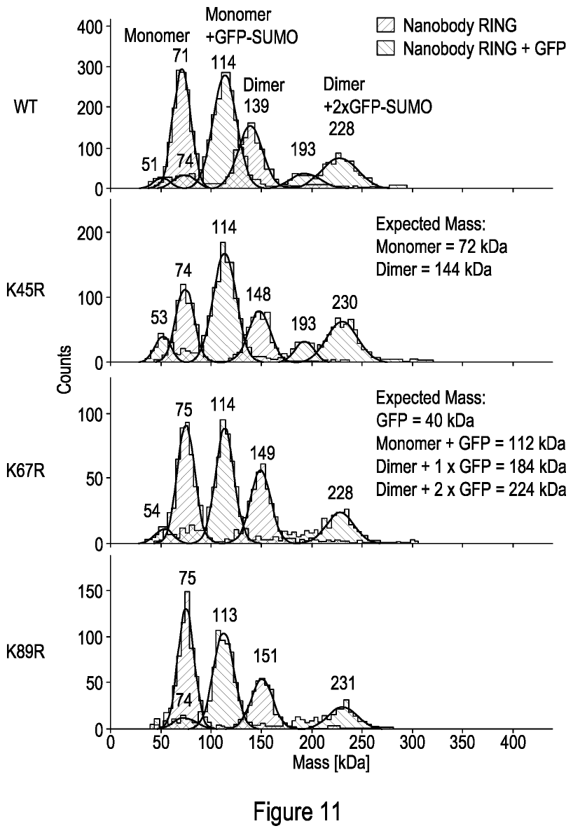

FIG. 11: Lysine to arginine mutations in the nanobody do not influence binding to GFP. MBP-GNb-RING alone (blue) or MBP-GNb-RING mixed with GFP-SUMO (orange) were analysed on a Refeyn mass photonics instrument to provide a mass distribution.

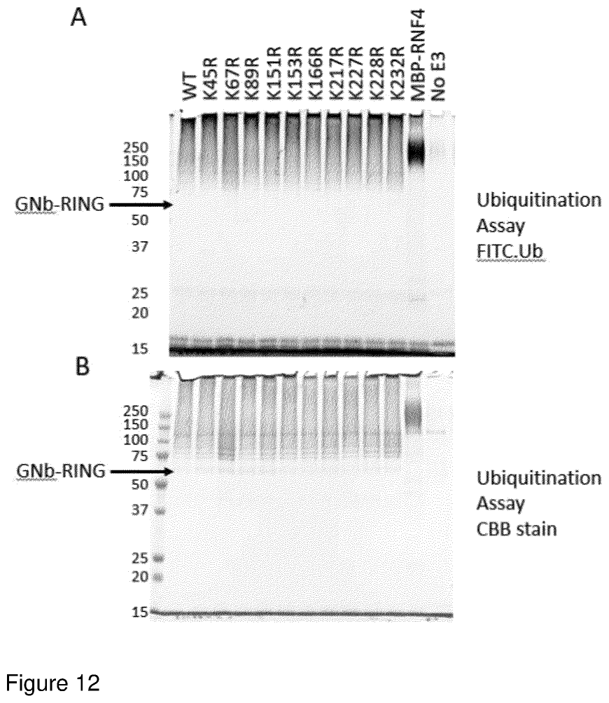

FIG. 12: Lysine to arginine mutations do not influence ubiquitination activity of MBP-GNb-RING. Purified proteins were assayed for ubiquitination in an in vitro assay containing ubiquitin E1 activating enzyme, ubiquitin conjugating enzyme Ubc5, FITC labelled ubiquitin and ATP. MBP-GNb-RING was added and the reaction allowed to proceed for 30 minutes at 37° C. The reaction was terminated by the addition of denaturing buffer and the products were fractionated by SDS-polyacrylamide gel electrophoresis and revealed by A. scanning for fluorescent ubiquitin or B. by staining of the gel with Coomassie Brilliant Blue.

### MATERIALS AND METHODS

### Plasmids

The coding sequences of a camelid-derived single-domain antibody (nanobody) (PDB accession 3K1K_C) raised against the green fluorescence protein (GFP), was generated synthetically (GeneArt, Thermofisher) with a 5′ HindIII and 3′ NheI restriction recognition sites. The coding sequence for residues 75-194, including the RING domain (residues 131-194) of Rattus norvegicus RNF4 (accession: NM_019182, UniProtKB—O88846) were amplified from previously generated expression constructs in pLou3 (Plechanovova et al., 2011) by the polymerase chain reaction (PCR) with 5′ NheI and 3′ BamHI-NotI restriction sites. The synthetically generated GFP nanobody and the RNF4 75-194 were ligated into the pCDNA5 FRT TO vector (Life Technologies) via a 3 point ligation HindIII-NheI-NotI, resulting in a GFP nanobody-wild-type RNF4 RING fusion (GNb-1xRING). To create a linear fusion of GNb-1xRING and the RING domain of RNF4 the RING domain was PCR- amplified with a 5′ BamHI sand a 3′ NotI restriction sites and inserted between the respective sites in GNb-1xRING and the resulting fusion was denoted “GNb-2xRING”. To generate nanobody-RING fusions targeting the NEDD8 specific protease NEDP1 (SENP8; accession NM_145204; UniProtKB—Q96LD8) the coding sequences for two nanobodies raised against this protein, nanobody 2 and NEDP1 nanobody 9 (REF), were produced by gene synthesis (GeneArt, Thermofisher) with 5′ HindIII and 3′ NheI restriction sites and sub-cloned into the pCDNA5 FRT TO-GNb-1xRING and pCDNA5 FRT TO-GNb-2xRING described above, replacing the GFP nanobody sequence and resulting in pCDNA5 FRT TO-NNb2-1xRING and pCDNA5 FRT TO-NNb9-2xRING, respectively. Subsequently, the coding sequences for RNF4 RING and RNF4 RING-RING containing M140A and R181A mutations within the RING domain sequences were PCR-amplified, starting from residue 131 as above, from previously generated constructs (Plechanovova et al., 2011) with 5′ NheI and 3′ NotI restriction sites and sub-cloned into the NNb2-1xRING and NNb9-2xRING constructs to replace the wild-type RNF4 RING sequences, resulting in in pCDNA5 FRT TO-NNb2-1xmtRING and pCDNA5 FRT TO-NNb9-2xmtRING, respectively. All nanobody-RING fusions contained an alanine-serine linker between the nanobody and the RNF4 sequence, and all nanoody-RING-RING fusion constructs contained a single glycine linker between the two RINGs. Bacterial expression constructs from all nanobody-RING and RING-RING fusions were created by PCR amplification of the fusion sequences from the above constructs with 5′ NcoI and 3′ XhoI sites and sub-cloned between the NcoI and SaII sites of pLou3 with N-terminal 6His-MBP tag and TEV protease cleavage site. To create a mammalian over-expression cDNA construct for Poly ADP ribose glycohydrolase (PARG; NM_003631; UniProtKB—Q86W56) with N-terminal enhanced yellow fluorescence protein (EYFP) tag we first created pEFIRES-P-EYFP-C1 by inserting the EYFP sequence after PCR-amplification from pEYFP-C1 (Invitrogen) with the upstream NheI site and adding in-frame 3′ SpeI and XhoI sites, into the Nhe I and Xhol I sites of the plasmid vector pEFIRES-P (Hobbs et al., 1998). We then PCR-amplified the PARG coding sequence was from cDNA clone MGC:57711, IMAGE:6064831 with 5′ SpeI and 3′ NotI restriction sites and cloned it into the respective sites of pEFIRES-P-eYFP-C1. RNF146 (NM_030963.2) and PEX10 (NM_002617.3) cDNA clones in pEFIRES-P-eYFP-C1 were obtained from the Medical Research Council Protein Phosphorylation and Ubquitilation Unit Reagents and services (https://mroppureagents.dundee.ac.uk/reagents-ccina-clones/overview) pCMV eYFP-IRESpuro PMLIII was kindly provided by Ellis Jaffray. All constructs were verified by DNA sequencing (dnaseq.co.uk).

### Cell Lines

HeLa and HEK293, (ATCC) were cultured in DMEM-Gltamax medium (Life Technologies 61965) supplemented with 10% Calf Serum and penicillin-streptomycin. Hela, Flp-in/T.rex cells (Life Technologies) were cultured in Minimum essential Medium—Eagle EBSS, with L-Glutamine (Lonza 12-611F) supplemented with 10% Calf Serum and penicillin-streptomycin. Hela Flp-in/T Rex (Life Technologies) grown in mono layer were transfected with each of the GFP or NEDP1 nanobody-wild-type or mutant RING/RING-RING fusion constructs descried above, along with the Flp recombinase vector pOG44, using Lipofectamine 3000 (Life Technologies) according to the manufacturers' instructions and selected with hygromycin at 100 μg/ml. Thereafter, stable cell populations were maintained in growth medium containing hygromycin (50 μg/ml) and blasticidin (5 μg/ml). Cells stably transfected with pCDNA5 FRT TO-GNb-1xRING or pCDNA5 FRT TO-GNb-2xRING were subsequently transfected with pEFRE-P-EYFP-C1-PARG or pCMV EYFP-IRESpuro PMLIII, respectively, selected with 1 μg/ml puromycin and maintained in growth medium containing puromycine (0.5 μg/ml), hygromycin (50 μg/ml) and blasticidin (5 μg/ml). Following confirmation of the YFP fusion protein degradation in response to doxycycline treatment homogeneous populations were selected by diluting the cell cultures to 1 cell/well and growing them under selection in 96-well plates until the appearance and growth to confluence of single colonies.

HEK293 cells stably expressing EYFP-SP100 were kindly provided by Ellis Jaffray. For doxycycline induction experiments cells were treated with 1 μg/ml doxycycline (Sigma). For experiments involving proteasome and/or autophay inhibition, 10 mMMG132 (Sigma; C2211), 1 μM Bortezomib (Selleckckem PS0341) or 100 nM Bafilomycin A1 (ENZO BML-CM110-0100), or a corresponding volume of DMSO was added to the medium 90 minutes prior to starting the experiment.

### siRNA Transfections

Cells were transfected with a pool containing an equimolar amount of four siRNA duplexes targeting NEDP1 (SENP8, accession: NM_145204, Dharmacon ON-TARGETplus; SENP8, 1—GAUCACGUCAGUUUCAUCA; SENP8, 2—UGAGUUACAUGGACAGUCU; SENP8, 3—CCAACAGUCAGUUUCAUGA; SENP8, 4—GGGAUGUACGUGAUAUGUA) to a final concentration of 10 nM, or a non-targeting control duplex (siNT) at the same concentration using Lipofectamine RNAiMAX (Life Tecnologies) according to the manufacturer's instructions. Total protein extracts were prepared 72 hours following transfection.

### Cell Lysis and Immunoblot Analysis

Cells were washed in PBS and whole-cell extracts were prepared by lysis in 2× Laemmli sample buffer (5% w/v SDS, 150 mM TRIS-HCl pH 6.7, 3) v/v glycerol, 0.01% w/v bromophenol blue) and heated at 95° C. for 5 mins. Protein concentration was measured using the Pierce BCA Protein Assay Kit (Thermo Fisher 23225) according to the manufacturers' instructions. Then b-mercaptoethanol was added to 700 mM and the cell lysates were separated in NuPAGE 4%-12% Bis-Tris gels (Thermo Fisher) and transferred to PVDF membrane. Primary antibody incubations were performed in PBS with 2% BSA and 0.1% Tween-20. For the secondary antibody incubations the 5% milk was used instead of the BSA. Primary antibodies used were mouse anti-GFP (Roche 11814460001, 1:1000), rabbit anti-NEDP1 (xx 1:1000), rabbit anti-NEDD8 (Epitomics 1571-1, 1:1000), sheep anti-RNF4 (homemade, 1:1000) and anti-alpha tubulin (ThermoFisher PA5-22060, 1:10000). HRP-coupled secondary anti-mouse, anti-rabbit and anti-sheep were purchased from Sigma. HRP-rabbit anti-camelid VHH was purchased from GenScript (A01681). The signal was detected by Pierce enhanced chemiluminescence (ThermoFisher 32106) and X-ray films.

### RNA Isolation and Quantitative RT-PCR

Total RNA was isolated using the E.Z.N.A Total RNA Kit (VWR R6834) with in-column DNase digestion following the manufacturer's protocol. cDNA was prepared using the First Strand cDNA Synthesis Kit (ThermoFisher K1612) and quantitative RT-PCR was performed using PerfeCTa® SYBR® Green (Quanta Bioscience) according to the supplier's protocol. qPCR was performed in either a 96 or 384-well format using Biorad CFX96/CFX384 or Applied Biosystems QuantstudioFlex 6 thermal cycler' Thermal cycling conditions were an initial denaturation step of 95° C. for 10 mins, and then 44 cycles of 95° C. for 15 secs, 60° C. for 60 secs followed by 95° C. for 10 secs and a melt curve of 65° C. to 95° C. The primers were designed to produce amplicons crossing the nanobody-RNF4 boundary. Standard curves were produced for each amplicon-specific primer set and for the used control gene Beta-2-Microglobulin (B2M) primers. RNA was always prepared from three independent cultures (replicates) representing each experimental condition and the PCR reaction was performed in duplicate for each RNA sample. The data were analyzed by the software accompanying the used instrument and presented after normalization against the control gene.

### High-Content Imaging

Cells were seeded in black, clear-bottomed 96-well plates (Greiner μClear) in 100 μl culture medium for 24 hours prior to the experiment.at the end of the experiment cells were washed twice with PBS, fixed with 4% formaldehyde and stained with 0.2 μg/ml DAPI (Thermofisher 62248) in combination HCS cellmask red stain (Thermofisher H32712). 100 μl of PBS was dispensed into wells and plates were sealed with an adhesive aluminium foil seal. Imaging was performed using an IN Cell 2000 microscope (GE Healthcare) to acquire three fields of view per well with a 10 or 20× lens (Nikon), capturing DAPI, CellMask and EYFP. Images were analysed using IN Cell Developer software (GE Healthcare), using a protocols designed to identify EYFP-PARG or EYFP-PML/EYFP-SP100 inclusions by region-growing or multi-scale top hat transformation, respectively. To measure EYFP nuclear intensity was used as the most robust parameter, while the measure of total organelle area per cell nucleus was selected as the most discriminatory for changes in EYFP-PML and EYFP-SP100 following treatment. For transfection efficiency calculations the cell/background intensity measure was found to give the most robust results and a threshold of 1.075 was used as the lower limit to be achieved by transfected cells. Data were obtained for >20000 cells per condition and the presented data represent the mean±sd. For degradation kinetics the time required to degrade 50% of the initial protein amount (t½) was deduced from the exponential resulting from plotting the obtained intensity or total area values against time.

### Protein Expression and Purification

Nanobody fusion proteins were expressed in E. coli SHuffle cells (New England BioLabs) at 20° C. overnight after induction with 0.1 mM IPTG. His6-MBP tagged fusion proteins were purified by Ni-NTA (Qiagen) affinity chromatography and dialyzed overnight in 50 mM Tris HCl pH7.5, 150 mM NaCl, 0.5 mM TCEP buffer. To remove the His6-MBP tag, fusion proteins were incubated with TEV protease, followed by Ni-NTA affinity chromatography to remove any uncleaved His6-MBP tagged proteins, the free His6-MBP tag and the TEV protease (also His6-tagged). Purified untagged Nanobody fusion proteins were then dialyzed against 50 mM Tris HCl PH 7.5, 150 mM NaCl 0.5 mM TCEP further purified by gel filtration (Superdex75) and flash-frozen in liquid nitrogen prior to storage at −80° C.

### Pull-Down Assay

The interaction between GFP nanobody Ring fusion proteins and GFP was studied using a pull-down experiment. His6-EGFP-SUMO1(20 μM) was incubated for ˜30 min at room temperature with RNF4 Ring-Ring fusion (negative control), Nanobody-Ring, Nanoboby-Ring-Ring or Mutants (20 μM) in a total volume of 200 ul containing 50 mM Tris.Cl pH7.5, 150 mM NaCl, 0.5 mM TCEP. 50 ul of Nickel beads were added in mixture and continue to incubate for 30 minutes. Nickel beads were collected on the bottom of the tube by centrifugation and samples were taken from the supernatant. Beads were washed 3 times with 0.5 ml of binding buffer. Bound proteins were eluted from the beads by addition of SDS-PAGE loading buffer and analyzed by SDS-PAGE.

In the pull-down experiments shown in FIG. 7A, His6-NEDP1(20 μM) was incubated for 5 min at room temperature with Ring-Ring (negative control), or NEDP1 nanobody2-2xRing or Nanobody2-Ring (˜20 μM) immobilized on Nickel beads (50 ul) in a total volume of 200 ul. Subsequently, beads were washed once as described above and bound material was eluted with SDA-PAGE loading buffer, analysed by SDS PAGE.

### Lysine Discharge Assay

UbcH5a˜Ub linked conjugate was prepared by mixing the following components for 20 min at 37° C.: 120 μM UbcH5a, 100 μM Ub, 0.2 μM Ube1, 50 mM Tris pH 7.5, 150 mM NaCl, 5 mM ATP, 5 mM MgCl2, 0.5 mM TCEP, 0.1% NP40. Apyrase (4.5 U ml-1, New England BioLabs) was then added to the reaction to deplete the ATP. The thioester was then mixed in a 1:1 ratio with test proteins, 10 mM L-lysine buffered with 50 mM Tris pH 7.5, 150 mM NaCl, 0.1% NP40, 0.5 mM TCEP. The final concentration of each component is about 30 μM thioester, 5 mM L-lysine, 50 nM fusion proteins. The reaction was incubated at room temperature, Samples were taken from the reaction mixture at the desired time points, mixed with non-reducing SDS-PAGE loading buffer and analyzed by SDS-PAGE.

### Microinjection

HeLa Flp-in/T.Rex cells stably expressing YFP-PML were seeded on to glass bottomed dishes (FluoroDish, WPI) and allowed to settle overnight. The cells were then microinjected with 30 μM GNb-2xRING mixed with an equal amount of mCherry (to localize the injected cells) in injection buffer (100 mM glutamic acid, pH 7.2 with citric acid (Izant et al., 1983), 140 mM KOH, 1 mM MgSO4 and 1 mM DTT) as described previously (Prescott et al., 1992). The cells were immediately transferred to the stage of a Zeiss LSM 710 confocal microscope with a 37° C. heated stage/chamber and 5% CO2 atmosphere and imaged by time-lapse. A z-stack of 7 images (6.3 μm depth) was taken at each time point and one stack was collected every 2 minutes. For each time point the z-stacks were compressed into a single maximum intensity projection and the time-lapse data was transferred into Imaris for quantitation. Injected cells were identified using the red, Texas Red channel and the Mean summed Green, GFP intensity of the injected cells was obtained following subtraction of 5000 units background, based on the uninjected surrounding cells, and plotted for each time point±sd.

### Electroporation of Cells with Nanobody-RING Fusions

Electroporation was performed using the Neon Transfection System (Thermo Fisher). Cells were washed with PBS and resuspended in Buffer R (Thermo Fisher) at a concentration of 8×107 cells/ml. We used 8×105 (10 μl) or 8×106 (100 μl) cells for selection by high content imaging or immunoblotting, respectively. Cells were mixed with 0.03 or 0.12 μg/μl, giving a final concentration of 0.375 pg or 1.5 pg of the recombinant fusion protein/cell, or PBS and electroporation was performed in 10 or 100 μl electroporation tips according to the manufacturers' instructions with 2 pulses at 1400V for 20 ms each. Cells were transferred to growth medium with10% FBS but without antibiotics. For immunoblot analysis aliquots were taken at the desired time points, and the reaction stopped by cooling on ice and centrifugation at 90×g for 10 min at 4° C. followed by cell lysis in 2× Laemmli sample buffer. For high content imaging aliquots were taken at the desired time point and the degradation reaction stopped immediately by adding the cells to an equal volume 8% formaldehyde in a black, clear-bottomed 96-well plates (Greiner μClear) followed by centrifugation at 90×g for 20 min at room temperature, washing, and DAPI staining as described. To determine transfection efficiency, cells were co-electroporated with a mCherry labelled protein and high content data were collected and analysed as described above.

### Quantitative Analysis of the Cellular Proteome Upon Induction of the NNb2-1xRING Construct

To monitor changes to the cellular proteome during induction of the NNb2-1xRING construct, a quantitative proteomics experiment was performed. Two cultures of HeLa Flp-in/T-REX NNb2-1xRING cells were grown in either ‘Light’ or ‘Heavy’ SILAC medium as described (Ong and Mann, 2007). Briefly, cells were grown in Dulbecco's modified Eagle's medium lacking all amino acids except L-lysine and L-arginine, which were supplemented with either isotopically typical lysine and arginine (‘Light’), or 13C615N2-lysine and 13C615N4-arginine (‘Heavy’). After full label incorporation, two 100 mm dishes of each labelled form of the cells were used for the SILAC comparisons shown in FIG. 4A. By this design, two parallel comparisons differing only by the SILAC labels could be used to monitor the effect of Dox treatment on the cellular proteome. After treatment with Dox or not, cells were washed twice with PBS and individual whole cell extracts were made by addition of 1.2× LDS sample buffer containing reducing agent (Invitrogen) followed by sonication and heating to 70° C. for 5 minutes. Protein concentrations were calculated by Bradford's method and 40 μg total protein was prepared for each SILAC mixture by mixing 1:1 (w:w) the appropriate extracts. These two mixes were fractionated by 4-12% acrylamide SDS-PAGE (Invitrogen NuPAGE Bis-Tris gels—MOPS buffer), and each lane of the Coomassie-stained gel excised into 16 equally sized slices (FIG. 4B). Gel pieces were subjected to in gel tryptic digestion (Shevchenko et al., 2006), employing both reduction with DTT and alkylation with chloroacetamide prior to digestion. Extracted peptides were dried down under vacuum and resuspended in 35 μL 0.1% TFA 0.5% acetic acid.

18 μL each peptide sample was analysed by LC-MS/MS on a Q Exactive mass spectrometer (Thermo Scientific) coupled to an EASY-nLC 1000 liquid chromatography system (Thermo Scientific) via an EASY-Spray ion source (Thermo Scientific). Peptides were fractionated on a 75 μm×500 mm EASY-Spray column (Thermo Scientific) over a 240 minute gradient. For all runs precursor ion full scan spectra were acquired over (m/z 300 to 1,800) with a resolution of 70,000 at m/z 400 (target value of 1,000,000 ions, maximum injection time 20 ms). Up to fifteen data dependent MS2 spectra were acquired with a resolution of 35,000 at m/z 400 (target value of 500,000 ions, maximum injection time 120 ms). Ions with unassigned charge state, and singly or highly (>8) charged ions were rejected. Intensity threshold was set to 2.1×104 units. Peptide match was set to preferred, and dynamic exclusion option was enabled (exclusion duration 15 s).

The 32 raw MS data files were processed using MaxQuant software (version 1.6.1.0) (Cox and Mann, 2008), and searched against UniProtKB human proteome (canonical and isoform sequences; downloaded in April 2013), plus a fasta file containing the sequence of the induced RING-NEDP1-nanobody construct:

The appropriate SILAC labels were selected and enzyme specificity was set to Trypsin/P (two missed cleavages). Importantly the re-quantify option was selected, without which peptides with missing SILAC counterpart peptides are not quantified and so proteins with large changes among conditions are not reported. This was necessary to obtain ratios for the nanobody construct itself. Carbamidomethylation of cysteines was set as a fixed modification and oxidation of methionines, acetylation of protein N-termini, and Gly-Gly adducts of lysines were set as variable modifications. Second peptide data was requested. The ‘match between runs’ option was selected to maximise the numbers of common identifications between the two SILAC mixes in identical or adjacent gel bands. Minimum peptide length was set to seven amino acids and a maximum peptide mass was 5,000 Da. A false discovery rate of 1% was set as a threshold at both protein and peptide level, and a mass deviation of 6 parts per million was set for main search and 0.5 Da for MS2 peaks. Slices were numbered 1 to 16 in the “Fraction” column of the experimental design template file, and all slices from the same SILAC mix were given the same ‘Experiment’ name to separate the ratio data into the two mixes (A & B).

The list of 5837 protein groups was filtered for entries from the decoy database, those identified by modified peptide(s) only, potential contaminants according to MaxQuant, and those with quantitative data in only one SILAC mix. This left 4506 proteins that could be compared between the two SILAC mixes. SigB values were calculated for each SILAC mix using Perseus (v 1.6.1.1) (Tyanova et al., 2016) using the ‘both sides’ method, truncated using a Benjamini-Hochberg FDR threshold of 0.05. Proteins ultimately defined as significantly affected by DOX treatment were those that met the SigB cutoff in both SILAC mixes and whose increase or decrease in response to DOX was consistent in both. This left four proteins.

For the slice-by-slice comparison in MaxQuant, every raw file was given a unique ‘Experiment’ name so protein data was separated by slice in the final output files. In this instance ‘requantify’ was turned off to ensure detection in any slice was not made by matching across files. All other MaxQuant setting were left as default.

Four peptides derived from NEDD8 itself were assigned by MaxQuant to the fusion protein NEDD8-MDP1 [UniProtKB-E9PL57 (E9PL57_HUMAN)]. One NEDD8 peptide not shared with this construct was assigned to NEDD8 [UniProtKB-Q15843 (NEDD8_HUMAN)]. For the slice-by-slice analysis, to extract protein level data for NEDD8 only, the five individual NEDD8 peptides intensity data were summed and these values entered into the proteinGroups table under the protein name “NEDD8 (MHT curated)”. This included data for the peptides (TLTGKEIEIDIEPTDKVER, EIEIDIEPTDKVER, IKERVEEKEGIPPQQQR, VEEKEGIPPQQQR, and ILGGSVLHLVLALR). The original entries for NEDD8-MDP1 and NEDD8 were deleted. In the non slice-by-slice (total protein change) analysis, the original entries were left as reported by MaxQuant due to there being no evidence of abundance change upon DOX treatment. Notably, peptides derived from the MDP1 portion of the NEDD8-MDP1 fusion were found exclusively in slice 14 in both mixes. As MDP1 itself has length 176 amino-acids and expected mass 20.1 kDa and slice 14 encompassed the 19-24 kDa region of the gel (FIG. 4B), this confirms NEDD8 and MDP1 peptides were falsely assigned to the NEDD8-MDP1 fusion protein rather than the individual proteins.

### Quantitative Analysis of the Cellular Proteome Upon Induction of the NNb2-1xRING Construct

To monitor changes to the cellular proteome during induction of the NNb2-1xRING construct, a quantitative proteomics experiment was performed. Two cultures of HeLa Flp-in/T-REX NNb2-1xRING cells were grown in either ‘Light’ or ‘Heavy’ SILAC medium as described (Ong and Mann, 2007). Briefly, cells were grown in Dulbecco's modified Eagle's medium lacking all amino acids except L-lysine and L-arginine, which were supplemented with either isotopically typical lysine and arginine (‘Light’), or 13C615N2-lysine and 13C615N4-arginine (‘Heavy’). After full label incorporation, two 100 mm dishes of each labelled form of the cells were used for the SILAC comparisons shown in FIG. 4A. By this design, two parallel comparisons differing only by the SILAC labels could be used to monitor the effect of Dox treatment on the cellular proteome. After treatment with Dox or not, cells were washed twice with PBS and individual whole cell extracts were made by addition of 1.2× LDS sample buffer containing reducing agent (Invitrogen) followed by sonication and heating to 70° C. for 5 minutes. Protein concentrations were calculated by Bradford's method and 40 μg total protein was prepared for each SILAC mixture by mixing 1:1 (w:w) the appropriate extracts. These two mixes were fractionated by 4-12% acrylamide SDS-PAGE (Invitrogen NuPAGE Bis-Tris gels—MOPS buffer), and each lane of the Coomassie-stained gel excised into 16 equally sized slices (FIG. 4B). Gel pieces were subjected to in gel tryptic digestion (Shevchenko et al., 2006), employing both reduction with DTT and alkylation with chloroacetamide prior to digestion. Extracted peptides were dried down under vacuum and resuspended in 35 μL 0.1% TFA 0.5% acetic acid.

18 μL each peptide sample was analysed by LC-MS/MS on a Q Exactive mass spectrometer (Thermo Scientific) coupled to an EASY-nLC 1000 liquid chromatography system (Thermo Scientific) via an EASY-Spray ion source (Thermo Scientific). Peptides were fractionated on a 75 μm×500 mm EASY-Spray column (Thermo Scientific) over a 240 minute gradient. For all runs precursor ion full scan spectra were acquired over (m/z 300 to 1,800) with a resolution of 70,000 at m/z 400 (target value of 1,000,000 ions, maximum injection time 20 ms). Up to fifteen data dependent MS2 spectra were acquired with a resolution of 35,000 at m/z 400 (target value of 500,000 ions, maximum injection time 120 ms). Ions with unassigned charge state, and singly or highly (>8) charged ions were rejected. Intensity threshold was set to 2.1×104 units. Peptide match was set to preferred, and dynamic exclusion option was enabled (exclusion duration 15 s).

The 32 raw MS data files were processed using MaxQuant software (version 1.6.1.0) (Cox and Mann, 2008), and searched against UniProtKB human proteome (canonical and isoform sequences; downloaded in April 2013), plus a fasta file containing the sequence of the induced RING-NEDP1-nanobody construct:

The appropriate SILAC labels were selected and enzyme specificity was set to Trypsin/P (two missed cleavages). Importantly the re-quantify option was selected, without which peptides with missing SILAC counterpart peptides are not quantified and so proteins with large changes among conditions are not reported. This was necessary to obtain ratios for the nanobody construct itself. Carbamidomethylation of cysteines was set as a fixed modification and oxidation of methionines, acetylation of protein N-termini, and Gly-Gly adducts of lysines were set as variable modifications. Second peptide data was requested. The ‘match between runs’ option was selected to maximise the numbers of common identifications between the two SILAC mixes in identical or adjacent gel bands. Minimum peptide length was set to seven amino acids and a maximum peptide mass was 5,000 Da. A false discovery rate of 1% was set as a threshold at both protein and peptide level, and a mass deviation of 6 parts per million was set for main search and 0.5 Da for MS2 peaks. Slices were numbered 1 to 16 in the “Fraction” column of the experimental design template file, and all slices from the same SILAC mix were given the same ‘Experiment’ name to separate the ratio data into the two mixes (A & B).

The list of 5837 protein groups was filtered for entries from the decoy database, those identified by modified peptide(s) only, potential contaminants according to MaxQuant, and those with quantitative data in only one SILAC mix. This left 4506 proteins that could be compared between the two SILAC mixes. SigB values were calculated for each SILAC mix using Perseus (v 1.6.1.1) (Tyanova et al., 2016) using the ‘both sides’ method, truncated using a Benjamini-Hochberg FDR threshold of 0.05. Proteins ultimately defined as significantly affected by DOX treatment were those that met the SigB cutoff in both SILAC mixes and whose increase or decrease in response to DOX was consistent in both.

For the slice-by-slice comparison in MaxQuant, every raw file was given a unique ‘Experiment’ name so protein data was separated by slice in the final output files. In this instance ‘requantify’ was turned off to ensure detection in any slice was not made by matching across files. All other MaxQuant setting were left as default.

Four peptides derived from NEDD8 itself were assigned by MaxQuant to the fusion protein NEDD8-MDP1 [UniProtKB-E9PL57 (E9PL57_HUMAN)]. One NEDD8 peptide not shared with this construct was assigned to NEDD8 [UniProtKB-Q15843 (NEDD8_HUMAN)]. For the slice-by-slice analysis, to extract protein level data for NEDD8 only, the five individual NEDD8 peptides intensity data were summed and these values entered into the proteinGroups table under the protein name “NEDD8 (MHT curated)”. This included data for the peptides (TLTGKEIEIDIEPTDKVER, EIEIDIEPTDKVER, IKERVEEKEGIPPQQQR, VEEKEGIPPQQQR, and ILGGSVLHLVLALR). The original entries for NEDD8-MDP1 and NEDD8 were deleted. In the non slice-by-slice (total protein change) analysis, the original entries were left as reported by MaxQuant due to there being no evidence of abundance change upon DOX treatment. Notably, peptides derived from the MDP1 portion of the NEDD8-MDP1 fusion were found exclusively in slice 14 in both. As MDP1 itself has length 176 amino-acids and expected mass 20.1 kDa and slice 14 encompassed the 19-24 kDa region of the gel (FIG. 4B), this confirms NEDD8 and MDP1 peptides were falsely assigned to the NEDD8-MDP1 fusion protein rather than the individual proteins.

### Targeted Proteomic Analysis of NEDP1

To monitor changes to NEDP1 levels in cells a Parallel Reaction Monitoring (PRM) (Peterson et al., 2012) method was employed. Tryptic peptide samples were prepared from parental Hela and cells expressing the ARMED construct for NEDP1 or GFP+/−doxycycline 1 ug/ml, as well as from recombinant NEDP1, and. To define a NEDP1 peptide inclusion list tryptic peptides derived from 500 ng of digested recombinant NEDP1 protein were analysed first in a data-dependent analysis (DDA) by LC-MS/MS on the Qexactive setup described above. Next, to define a list of cellular peptides to control for sample loading in the PRM analysis, a mixed sample was generated by pooling tryptic peptides from PARENTAL, NEDP1, and GFP control cell lines+/−doxycycline, and 1 ug was run in triplicate immediately following the recombinant samples. iRT peptides were spiked into all samples (Biognosys Cat #Ki-3002-2), and both the iRT and control peptides were added to the inclusion list. MS runs were acquired over identical 90 minute gradients with flow rate 20 ul/min, buffer A HPLC-grade water 0.1% formic acid, and buffer B mass spectrometry-grade acetonitrile 0.1% formic acid. DDA methods consisted of precursor ion full scan acquired over m/z range of from 300 to 1,800 with a resolution of 70,000 at m/z 200, a target value of 1,000,000 ions, and maximum injection times of 20 ms. Up to 4 data dependent MS2 spectra were acquired with a resolution of 70,000 at m/z 200, a target value of 1,000,000 ions, and a maximum injection time 300 ms. Ions with unassigned charge state, and singly or highly (>8) charged ions were rejected. Intensity threshold was set to 2.0×10\hat{ }4 units. Peptide match was set to preferred, and dynamic exclusion to 40 s. The run was conducted in positive ion mode.

NEDP1-nanobody, iRT peptides, and the UniProtKB human proteome (canonical and isoform sequences; downloaded in April 2013) using 1% FDR for both proteins and peptides, trypsin digestion with 4 max missed cleavages, minimum peptide length of 5 amino acids, and maximum peptide mass of 10,000 Da. Calculate Peak Properties was selected, a threshold score of 40 was applied, and all other settings left as default. The inclusion list combined 24 NEDP1 peptides and 11 iRT peptides, as well as 63 high scoring human protein peptides for use as sample loading controls.

PRM was performed on 12 ul (approximately one third) of each of the 18 cellular peptide samples described above, each spiked with iRT control peptides, using the same 90 min elution gradients as the DDA runs. PRM methods included precursor full scans acquired over a scan range of 300-1800 m/z with chromatogram peak widths of 30 s, resolution 70,000 at 200 m/z, a target value of 1,000,000, and a maximum injection time of 100 ms. The inclusion list generated from the DDA data was imported. Up to 12 data dependent MS2 spectra were acquired with a resolution of 70,000 at m/z 200, a target value of 200,000 ions, a maximum injection time of 247 ms, NCE 28, and spectrum data type was set to centroid.

MSConvertGUI v3.0.18270-f64d6f0fe was used to convert PRM .raw files to .mzXML/.wiff format for Skyline analysis. Filters was set to Peak Picking and MS levels was set to 1-2, otherwise settings were left at default.

A blank Skyline document was generated with default settings except where noted in below. A redundant library was kept and a set of 11 Biognosys iRT peptides was used (setting Biognosys-11 iRT-C18). The cut-off score was set at 95, corresponding to a FDR of 5%. The reported iRT graph contained 9 points, with slope 1.7140, intercept −58.1619, and R-squared value 0.993. iRT standard values were recalibrated relative to the peptides added, with a time window of 5 minutes. The sequence of the recombinant NEDP1protease, which contained an additional GA at the N-terminus as a result of the TEV cleavage, was added to the target list, as were the inclusion list peptides in FASTA format. The inclusion list contained 100 peptides, 9 of which were sequence duplicates of other inclusion list peptides but differed by charge. Digestion enzyme was set to Trypsin [KR|P], with 1 max missed cleavage. Background proteome was the human proteome plus NEDP1 with GA inserted at the N-terminus, digested with trypsin with 1 maximum missed cleavage. The minimum peptide length searched for was 5 amino acids and maximum was 25. Variable modifications selected were carbamidomethylation of cysteines, oxidation of methionine, acetylation at the N-terminus, and carboxymethylation at the N-terminus. Precursor charges was set to 2-5, ion charges was set to 1-5, and ion types was set to y,b,p. Skyline was set to pick 20 product ions, with a minimum of 5. Minimum m/z was set to 300 and maximum to 1800. Under MS1 filtering the isotope peaks included was set to count, and precursor mass analyser was set to Orbitrap. Resolving power was set to 70,000 at 200 m/z. Under MS/MS filtering, the acquisition method was set to targeted, and the product mass analyser was set to Orbitrap. Resolving power was set to 70,000 at 200 m/z. Only scans within 5 minutes of MS/MS IDs were used. 18 PRM .wiff files were imported with sample numbering scheme identical to above, empty proteins and peptides were removed and minimum DOTP threshold was set to 0.75 for NEDP1 peptitde analysis. Some chromatogram peak boundaries reported by Skyline were empirically observed to be in error and were manually adjusted. In these instances, the original boundary is shown in the individual Skyline sample chromatograms by magenta shading and the adjusted boundary is indicated by dashed lines. Analysis of the loading normalisation sample resulted in 63 high scoring peptides from 60 proteins which were added to the inclusion list. PRM MS1 peak intensities corresponding to 34 of these peptides were averaged to generate correction factors for sample loading errors. Selection of appropriate sample as well as positive control peptides was based on points across peak >7, mass error <4 ppm, and idotp >0.75. Median number of points across peak for all sample and control peptides was 16.

The 3 NEDP1 peptides detected in the MS2 analysis were LAFVEEK, LEAFLGR, and QVAEKLEAFLGR; however, no QVAEKLEAFLGR fragment ions were detected in the NEDP1 ARMeD construct plus doxycycline cells. To calculate fold-depletion of NEDP1 upon doxycycline induction of the nanobody-ring fusion, 7 fragment ions from the LAFVEEK and LEAFLGR peptides were analysed. The sums of all fragment intensities from each replicate were calculated. For each set of triplicate samples, the median of these sums was determined. We define the fold-depletion as the ratios of these means, which were taken for each of the following pairwise comparisons: PARENTAL+/−, NEDP1−/PARENTAL−, NEDP1+/PARENTAL−, GFP+/PARENTAL−, GFP−/PARENTAL−, and NEDP1+/ NEDP1−. P-values were calculated via two-tailed unpaired t tests using Prism software v8.1.2.

After knockdown the NEDP1+Dox chromatogram peak areas were near background which introduced some challenges in their analyses. When chromatogram peaks were partially overlapped by adjacent spurious peaks the Skyline default was to report N/A instead of an area value. Boundaries for the LEAFLGR peaks in samples 1, 2, 12, and 17 were manually adjusted. This had the effect of avoiding the default N/A and instead reporting a value that was inflated by the adjacent spurious peak. The LAFVEEK peptide y4 ion had an adjacent spurious peak with a significantly different mass (>20 ppm), and the LEAFLGR peptide y6 ion had an adjacent spurious peak of nearly identical mass. Given that these areas are inflated by the presence of adjacent spurious peaks, any fold-reduction value generated for the knockdown by boundary adjustment as per above will be skewed below the true value.

Only 5 points across peak (PAP) were reported for the sample 12 LEAFLGR peptide, and 2 of the peaks were empirically observed to be indistinguishable from background. Despite being below the threshold of 7, the data was included in order to permit the calculation of a baseline magnitude for the knockdown.

LAFVEEK, LEAFLGR, and QVAEKLEAFLGR peptide sequences were blasted against the human proteome (taxid 9606) using NCBI Blast: Protein Sequence to verify uniqueness. All LAFVEEK, LEAFLGR, and QVAEKLEAFLGR 100% query cover/100% sequence identity matches were unique to NEDP1. NEDP1 protein was queried on phosphosite.org and was found to be potentially acetylated at lysine 146. We were able to detect the relevant peptide (LAFVEEK) and do not expect presence of doxycycline to affect acetylation levels. The reduction in abundance of this peptide upon addition of doxycycline matches that of the LEAFLGR and QVAEKLEAFLGR peptides, which are not known to be acetylated. Samples were not run in a blinded fashion.

### RESULTS

### Antibody-RING-Mediated Destruction (ARMeD)

The ubiquitin E3 ligase RNF4 contains a C-terminal RING domain responsible for dimerization and recruitment of the ubiquitin loaded E2 conjugating enzyme, while the N-terminal region contains 4 SUMO Interaction Motifs (SIMs) that allow the E3 ligase to engage substrates containing multiple SUMOs (FIG. 1A). When a SUMO modified substrate is bound and ubiquitin loaded E2 is primed for catalysis (Dou et al., 2012; Plechanovova et al., 2012; Pruneda et al., 2012) a nucleophile (usually the ε-amino group of lysine) attacks the thioester bond linking the ubiquitin to the active site of the E2 and ubiquitin is transferred to substrate (FIG. 1A). To allow the E3 ligase to be used against any defined target we sought to change the substrate recognition properties of RNF4. The SUMO recognition domain was therefore replaced with a camelid nanobody that could direct the RING domain of RNF4 (nanobody-1xRING) to the target of the nanobody. We also generated a constitutively dimeric form of RNF4 (nanobody-2xRING) by linking the nanobody to two copies of the RNF4 RING connected by a short linker (Branigan et al., 2015; Plechanovova et al., 2012; Rojas-Fernandez et al., 2014). The nuclear localization signal (NLS) of RNF4 was retained in all constructs to allow efficient targeting of nuclear proteins. Initially, we used a well-characterised nanobody raised against Green Fluorescent Protein (GFP) that also recognises Yellow Fluorescent Protein (YFP) (Kirchhofer et al., 2010). These constructs were used to generate Hela Flp-in/T Rex cells where expression of the GFP-nanobody RING fusions was doxycycline (Dox) dependent (FIG. 1B-D). Our expectation was that expression of a nanobody RING fusion in cells would lead to ubiquitin-proteasome mediated degradation of the target protein. We describe this process as Antibody-RING-Mediated Destruction (ARMeD). To test this hypothesis, we stably expressed YFP-PARG (Poly ADP ribose glycohydrolase) in the Hela Flp-in/T Rex cells already expressing the Dox inducible GFP-nanobody RING fusions. Western blotting indicated that after Dox induction of the GFP-nanobody RING (GNb-1xRING), YFP-PARG was no longer detectable by Western-blotting (FIG. 1E). Fluorescence imaging also revealed that Dox induction led to depletion of PARG in almost all cells (FIG. 1F), while high content analysis of the generated images revealed that Dox induction led to a 19 fold reduction in YFP-PARG levels (FIG. 1G). To distinguish between the two main modes of ubiquitin dependent degradation, Dox induction was carried out in the presence of the autophagy inhibitor bafilomycin A1 or proteasome inhibitors MG132 or bortezomib. Western blotting (FIG. 1H) and high content imaging (FIG. 1I) indicated that GNb-1xRING induced degradation of YFP-PARG was unaffected by bafilomycin, but was blocked by both MG132 and bortezomib. Thus, the GFP-nanobody RING induced degradation of YFP-PARG via the ubiquitin proteasome system. To establish the time course of degradation GNb-1xRING was induced by Dox and YFP-PARG expression was monitored by Western blotting (FIG. 1J) over a 12 hour period or by high content imaging (FIG. 1K) over a 24 hour period. The t½ determined from the quantitative imaging data was 7.1 hours. While YFP-PARG is a soluble nuclear protein, a more demanding test of the utility of the GFP-nanobody RING was its ability to induce degradation of YFP-PML (Promyelocytic Leukaemia) protein that is located in nuclear bodies and is stabilised in these bodies by a dense network of SUMO-SIM interactions (Shen et al., 2006). Cells expressing YFP-PML and a Dox inducible GFP-nanobody 2xRING (GNb-2xRING) were generated. Western blotting indicated that after Dox induction of GNb-2xRING, YFP-PML was no longer detectable (FIG. 2A). Fluorescence imaging also revealed that Dox induction led to depletion of PML in almost all cells (FIG. 2B) while high content analysis of the images revealed that Dox induction led to a 9 fold reduction in YFP-PML levels (FIG. 2C). Western blotting (FIG. 2D) and high content imaging (FIG. 2E) indicated that while GNb-2xRING induced degradation of YFP-PML was not inhibited by bafilomycin, it was blocked by both MG132 and bortezomib. Thus, GNb-2xRING also induces degradation of YFP-PML via the ubiquitin proteasome system. Time course analysis of the degradation of YFP-PML over a 25 hour period monitored by Western blotting (FIG. 2F) or over a 24 hour period analysed by high content imaging (FIG. 2G) showed the t½ to be slightly longer than for YFP-PARG at 10.1 hours. However, we note that the degradation curves for YFP PARG and YFP-PML were an imperfect fit to the exponential equations used which could be due to the delayed onset of degradation and, therefore, the actual t½ may be even shorter than the one calculated on the basis of this equation for both proteins.

While targeting the above two proteins was likely to succeed due to the presence of the functional nuclear localization signal (NLS) of RNF4, we also proposed that the nanobody-RING fusions might also be active while on transit from their cytoplasmic synthesis site to the nucleus. To test this hypothesis we generated Hela Flp-in/T Rex cells expressing YFP-tagged E3 ubiquitin-protein ligase RNF146 or YFP-tagged PEX10 (peroxisomal biogenesis factor 10) in addition to Dox inducible GFP-nanobody 1xRING (GNb-1xRING). Doxycycline induction of the GNb-2xRING fusion in those cell lines led to a 3-4 fold reduction of the targeted YFP-tagged proteins. We conclude that our GFP-nanobody-RING fusions can be used for targeting both nuclear and cytosolic proteins.

### Antibody-RING-Mediated Destruction (ARMeD) of Endogenous NEDD8 Specific Protease NEDP1

To explore the application of ARMeD to endogenous, unmodified proteins it was necessary to generate protein-specific nanobodies. The NEDD8 specific protease NEDP1 (Mendoza et al., 2003), has been structurally resolved (Shen et al., 2005) and the consequences of its depletion previously established (Maghames et al., 2018). We generated a series of camelid nanobodies against NEDP1, among which nanobody 2 and nanobody 9 both bind to NEDP1 and inhibit its catalytic activity (to be described in detail elsewhere). To test their activity in vivo Nanobody 2 was fused to single RING of RNF4 (NNb2-1xRING) while Nanobody 9 was fused to a constitutively dimeric form of RNF4 (NNb9-2xRING). Nanobody 2 was also fused to single RING of RNF4 inactivated by the double mutation M140A, R181A (Plechanovova et al., 2011) (NNb2-1xmtRING) while Nanobody 9 was fused to a similarly mutated constitutively dimeric form of RNF4 (NNb9-2xmtRING). The mutated residues correspond to M136 and R177 in human RNF4 but the RING domain sequence is identical in both orthologs. These constructs were used to generate Hela Flp-in/T Rex cells where expression of the NEDP1-nanobody RING fusions was doxycycline (Dox) dependent. Expression of the fusions was induced by Dox treatment for 24 hours, while cells treated with a pool of siRNAs to NEDP1 or non-targeting controls for 48 hours were used for comparison. Analysis by Western blotting revealed that after Dox treatment NNb2-1xRING, but not its inactive mutant counterpart induced the degradation of NEDP1to undetectable levels (FIG. 3A). In comparison, siRNA reduced the level of NEDP1, but depletion was incomplete and NEDP1 could still be detected. Even before application of Dox, NEDP1 levels were reduced in cells containing the NNb9-2xRING construct. After Dox treatment NEDP1 levels were reduced to undetectable levels. Again, mutational inactivation of the RING blocked NEDP1 degradation. In all situations, apart from NNb9-2xRING, Dox induction resulted in the accumulation of the nanobody-RING fusions at the correct molecular weight. In the case of NNb9-2xRING, NEDP1 degradation is apparent even in the absence of Dox. This is due to leaky, Dox independent expression as determined by RT-PCR. As the fused RINGs create a hyperactive E3 ligase even the small amount produced under these conditions results in substantial NEDP1 depletion. After Dox induction NEDP1 is undetectable by Western blotting but the NNb9-2xRING fusion is also undetectable. This is likely due to autoubiquitination of the E3 ligase as the mutated, inactive form is detected and mRNA encoding NNb9-2xRING is induced by Dox.

NEDP1 depletion with NNb2-1xRING or NNb9-2xRING leads to the accumulation of NEDD8 conjugates and the appearance of NEDD8 dimers (FIG. 3A). When NEDP1 is depleted with SIRNA NEDD8 dimers and higher molecular weight conjugates are only modestly increased. Counterintuitively, although NEDP1 is not depleted, expression of NNb2-1xmtRING NNb9-2xmtRING leads to accumulation of NEDD8 modified species (FIG. 3A). This is explained by the direct inhibition of the activity of NEDP1 by the nanobodies, even though NEDP1 is not turned over. To establish the time course of degradation of NEDP1, NNb2-1xRING was induced by Dox and NEDP1 expression was monitored by Western blotting. NEDP1 levels decreased with time and NEDP1 was undetectable after 12 hours (FIG. 3B).

While NNb2-1xRING and NNb9-2xRING reduce NEDP1 to undetectable levels by Western-blot we used the highly sensitive mass spectrometry technique Parallel Reaction Monitoring (PRM) (Peterson et al., 2012) to obtain quantitative analysis of the scale of NEDP1 depletion. Three well resolved peptides from NEDP1 were selected for analysis, and for each peptide a number of fragment ions were quantified (FIG. 3C, D). Combining the data for the three peptides indicates that NNb2-1xRING reduces NEDP1 levels by at least 8 fold, and some NEDP1 peptide fragments become undetectable even by this method upon DOX treatment, and therefore cannot contribute to the final calculations (FIG. 3E).

### Selectivity of ARMeD

The target specificity of the ARMeD approach was evaluated by shotgun proteomic analysis of crude cell lysates from cells containing the Dox inducible NEDP1 nanobody fused RING (NNb2-1xRING). A SILAC (Mann, 2006) approach was taken whereby cells treated with vehicle only were grown in ‘Light’ medium while cells induced to express NNb2-1xRING by Dox treatment were grown in ‘Heavy’ medium (FIG. 4A). Whole cell extracts were prepared, mixed in a 1:1 ratio (MIX A) and fractionated by SDS-PAGE (FIG. 4B). The gel was cut into 16 slices and each slice subjected to in-gel trypsin digestion and the eluted peptides analysed by mass spectrometry. To increase the robustness of the analysis a label swap experiment was conducted where vehicle treated cells were grown in heavy isotopes and Dox treated cells were grown in normal medium (MIX B). The data from both mixes were analysed in MaxQuant and the Log2 H/L ratios displayed on a scatter plot (FIG. 4C). Of the 4600 proteins detected in all 4 SILAC conditions the only protein to show a consistent change after Dox induction was the NNb2-1xRING fusion protein (FIG. 4C-E). While NEDP1, the target for degradation, was detected in the vehicle treated samples, it was not detected in Dox treated cells, although the previous PRM approach had determined that Dox induction reduced its level by 8 fold (FIG. 3C-D). As NEDP1 depletion leads to an accumulation of NEDD8 conjugates (FIG. 3A) we analysed the distribution of NEDD8 peptides in each of the gel slices. This revealed that Dox induction led to a decrease in the intensity of NEDD8 peptides in the region of the gel containing free NEDD8 and a general increase in the intensity of NEDD8 peptides in regions of the gel representing proteins with a higher apparent molecular weight. However the region of the gel containing NEDD8 modified Cullins was unaffected after NEDP1 depletion (FIG. 4E). Furthermore, the NNb2-1xRING construct itself also displayed higher molecular weight forms upon induction (FIG. 4F), consistent with a mechanism of self-ubiquitination as described above. Thus the nanobody directed RING fusion displays remarkable specificity for its target protein.

### Acute and Rapid Degradation of Target Proteins by Purified Nanobody-RING Fusions

Although much faster than indirect nucleic acid-based methods for protein manipulation, Dox induced nanobody-RING fusions act over a timescale of hours. This will include time taken for the chemical to be absorbed into cells and the construct itself to be expressed in quantities required to degrade the target. Furthermore, the DOX-inducible system also requires genetic manipulation of the cell population. To circumvent these issues and to attempt to hasten protein degradation we decided to directly introduce purified Nanobody-RING fusion proteins into cells. Thus GNb-1xRING, GNb-2xRING and their inactive RING counterparts (containing the M140A, R181A double mutation) GNb-1xmtRING, GNb-2xmtRING were expressed in bacteria and purified to homogeneity (FIG. 5A). To confirm that the purified proteins retained target binding and E3 ligase activity we carried out in vitro experiments. GNb-1xRING, GNb-2xRING, GNb-1xmtRING and GNb-2xmtRING, but not an RNF4 fused RING alone, efficiently pulled down a 6His-GFP-SUMO1 fusion protein (FIG. 5B). Ubiquitin E3 ligase activity was tested in a substrate independent fashion using a lysine discharge assay (Branigan et al., 2015), that measures the ability of the RING to activate the ubiquitin˜Ubc5 thioester bond to nucleophilic attack by free lysine. The RNF4 fused RING alone and the GNb-2xRING, but not the GNb-2xmtRING, were active in lysine discharge activity (FIG. 5C). To test the activity of the Nanobody-RING fusion proteins in vivo we used microinjection to introduce purified GNb-2xRING into cells expressing YPF-PML. Microinjected cells were marked by co-injection of an mCherry protein (FIG. 5D) and the fluorescent images were collected in real time. Quantitation of the YFP signal from PML revealed that the protein was degraded with a t½ of 10.9 minutes (FIG. 5E). While microinjection demonstrates the principle that purified GNb-2xRING can be used as a single component reagent to induce target protein degradation, we sought to extend this to rapid, time resolved degradation in bulk populations of cells. A variety of methods were therefore tested for the simultaneous delivery of GNb-2xRING to large numbers of cells. As a transfection efficiency control mCherry was included with GNb-2xRING. Neon electroporation proved to be the most satisfactory approach. Using high content imaging we could demonstrate that 1.5 pg of electroporated protein/cell resulted in a high proportion (>80%) of cells displaying mCherry fluorescence above background levels (FIG. 5F). To assess target degradation GNb-2xRING was electroporated into cells expressing the PML body component SP100 as a YFP fusion protein. High content imaging was used to evaluate the extent of degradation of YFP-SP100 after 60 minutes. Using only 0.375 pg of GNb-2xRING/cell little degradation was observed, but with 1.5 pg of GNb-2xRING/cell SP100 levels were reduced by 85% (FIG. 5G). The time taken for degradation of YFP-SP100 was determined by electroporating cells with purified GNb-2xRING and cells processed for high content imaging or collected for Western blotting at various times post-electroporation. Western blotting indicates that YFP-SP100 is efficiently degraded by 30 minutes, while high content imaging indicates that maximal degradation has been reached 10 minutes after electroporation. Thus purified preparations of Nanobody-RING fusions can be used as a reagent to rapidly degrade target proteins in bulk populations of cells.

### Rapid Antibody-RING-Mediated Destruction of Endogenous, Unmodified NEDP1

While we have established that purified GNb-2xRING can induce rapid degradation of a YFP modified protein in a large population of cells, the ultimate test of ARMeD is the demonstration that it can induce the rapid degradation of endogenous, unmodified protein targets. We therefore evaluated the ability of the NEDP1 protease specific nanobody-RING fusions to induce the rapid degradation of NEDP1 in bulk populations of HEK293 cells. NNb2-1xRING and NNb2-2xRING were expressed in bacteria and purified to homogeneity. To confirm that the purified proteins retained their biological activities of binding to NEDP1 and E3 ligase activity, in vitro experiments were conducted. NNb2-1xRING and NNb2-2xRING, but not an RNF4 fused RING alone, efficiently pulled down a 6His-NEDP1 protein (FIG. 6A). Ubiquitin E3 ligase activity was tested in a lysine discharge assay as described above. The RNF4 fused RING alone and the NNb2-2xRING, displayed comparable E3 ligase activity but the NNb2-1xRING, was less active. (FIG. 6B). The ability to degrade endogenous, unmodified NEDP1 was determined by electroporating cells with either purified NNb2-2xRING or NNb2-1xRING and cells collected at various times post-electroporation. Western blotting indicates that NNb2-2xRING efficiently induces the degradation of NEDP1 by 30 minutes (FIG. 6C), whereas degradation induced by NNb2-1xRING was less efficient (FIG. 6D). The phenotypic output of cells depleted for NEDP1 is the appearance of NEDD8 dimers and the accumulation of NEDD8 modified proteins. NNb2-2xRING and NNb2-1xRING both induce the appearance of NEDD8 dimers and the accumulation of higher molecular weight NEDD8 modified species (FIG. 6C&D). Thus with the appropriate Nanobody-RING fusion, unmodified, endogenous cellular proteins can be rapidly targeted for degradation using purified preparations of the Nanobody-RING fusion in bulk populations of cells.

### DISCUSSION

Conventional gene knockouts and RNAi are widely used approaches to analyse the biological function of proteins. However the disadvantages of these approaches are that it takes a long time (days) to effectively ablate mRNA expression and once translation of the target protein has ceased depletion of the protein is entirely dependent on its inherent half-life. In fact many components of essential cellular structures, such as the nuclear pore complex are stable over many months (Toyama et al., 2013) and would thus be resistant to depletion using methods that target RNA or DNA. Likewise protein aggregates that are the hallmark of neurological disease are stable over years. Here we describe Antibody-RING Mediated Destruction (ARMeD) as a route to circumventing these problems. In this approach the RING domain of RNF4 is fused to a nanobody to create a small ubiquitin E3 ligase with unique target specificity that can be used to target the protein recognised by the nanobody for ubiquitin proteasome mediated destruction. These small proteins can be expressed in bacteria and purified in high yield to provide a reagent that, as a single component, can be introduced into cells to induce degradation of the target protein within minutes and with minimal off-target degradation (FIG. 4). We envisage two distinct modalities for the use of ARMeD. In the first approach the RING domain could be fused to one of the many nanobodies available to mediate destruction of the target protein. A recent analysis indicated that almost 800 single domain antibodies, or nanobodies, have been characterised and the sequences made available to the scientific community (Wilton et al., 2018). This number is increasing rapidly as these nanobodies are being used in applications including structural biology (Pardon et al., 2014), super-resolution microscopy (Pleiner et al., 2015) and intracellular signalling studies (Prole and Taylor, 2019). As coverage of the proteome increases and the nanobody database expands it should be possible to search the database for a nanobody to the protein to be targeted, have a G-block synthesised corresponding to the sequence of the nanobody and have the nanobody-RING fusion expressed in bacteria and purified ready for knockdown studies in a matter of days. The advantage of this approach is that protein depletion can be achieved without any prior manipulation of the cells under study. However when nanobodies of the target protein are not available an alternative approach is to either use a pre-existing cell line containing a GFP tagged protein, or to generate an endogenously GFP tagged protein using CRISPR/cas9 technology. The GNb-2xRING nanobody-RING fusion could then be used as a single reagent to induce the degradation of any GFP tagged protein. This could be done in almost any eukaryotic organism as the RING domain of RNF4 is highly conserved and human RNF4 was shown to function in yeast (Sun et al., 2007). As the ARMED system appears to display minimal off-target destruction, target selection is dependent on the unique specificity of the nanobody. This represents a major advantage of the nanobody based approach as the system is capable of selective degradation of post-translationally modified proteins (Chirichella et al., 2017) or the mutant proteins (oncogenes) responsible for cancer (Quevedo et al., 2018). While considerable challenges remain to be overcome in the delivery of proteins, the therapeutic application of the ARMED approach may have utility in the destruction of disease-causing proteins.

### Modified E3 Ligases

One of the drawbacks of using ubiquitin E3 ligases to target substrates for degradation is that the E3 ligases themselves are ubiquitinated in a process known as autoubiquitination. This leads to self-destruction of the E3 ligase and is elevated in the absence of substrate. Without being bound by theory, it is thought that this might act in a regulatory fashion as it could get rid of E3 ligase that was no longer needed.

As ubiquitination takes place on lysine residues a possible approach to increase the stability of the E3 ligase is to change the lysine residues in the E3 ligase to arginine. This is a conservative mutation that retains the positive charge on the protein but yields a more stable E3 ligase molecule as arginine residues cannot be ubiquitinated.

However lysine residues may play an important role in the catalytic mechanism of the E3 ligase or may be important for substrate recognition. Before generating GNb-RING with 10 lysine to arginine mutations, the effect of any lysine to arginine mutations on the function of the E3 ligase was determined.

Analysis of the crystal structure of the RING domain of RNF4 bound to ubiquitin loaded E2 (FIG. 9A) revealed that none of the 7 lysine residues in the RING made important contacts that might be disrupted by mutation. Likewise inspection of the complex between the GFP nanobody and GFP indicated that none of the lysine residues made important contacts (FIG. 9B). To test the role of each lysine residue a series of GNb-RING proteins were generated in which individual residues were changed to arginine. This was done in the context of a Maltose Binding Protein (MBP) GNb-RING fusion protein (FIG. 10A) that could be expressed in and purified from bacteria. In addition to the wild type (WT) protein, three lysine to arginine mutations where introduced to the GNb nanobody (K45R, K67R and K89R) and seven lysine to arginine mutations to the RING domain (K151R, K153R, K166R, K217R, K227R, K228R and K232R) that were expressed in bacteria and purified (FIG. 10B). To determine binding to target we mixed MBP-GNb-RING (expected monomer mass 72 kDa) with GFP-SUMO (expected mass 55 kDa) and analysed complex formation by mass photometry (Young et al., 2018). WT MBP-GNb-RING alone (FIG. 11, blue) shows that as expected a mixture of monomer (71 kDa) and dimer (139 kDa) are present and when GFP-SUMO is added the monomer binds a single GFP-SUMO shifting is mass from 71 to 114 kDa, while the dimer predominantly binds two molecules of GFP-SUMO, shifting is mass from 139 to 228 kDa (FIG. 11, orange). Essentially the same pattern is observed for the K45R, K67R and K89R mutants indicating that mutating these lysine residues does not alter binding to its target protein. To determine the intrinsic ubiquitin E3 ligase activity of lysine to arginine mutations in GNb-RING the MBP-GNb-RING fusions were assayed for ubiquitination activity in an in vitro assay containing fluorescently labelled ubiquitin (FITC. Ub). MBP is a large protein (45 kDa) containing many lysine residues that can be ubiquitinated by the attached RING. Mutation of a single lysine residue is unlikely to affect the amount of ubiquitin incorporated unless the lysine residues affects the intrinsic E3 ligase activity of the RING. As FITC. Ub is a small protein (7 kDa) it migrates with the dye front on the gels, but if it is linked to MBP-GNb-RING in an autoubiquitination event it will migrate at 75 kDa or above. It is apparent from FIG. 12A that high molecular mass ubiquitin accumulates in WT and all of the lysine to arginine mutants. Thus none of the lysine to arginine mutant affects intrinsic ubiquitination activity of the RING. This is also evident from inspection of the gel stained for protein with Coomassie Brilliant Blue dye (FIG. 12B). In conclusion, none of the lysine to arginine mutations in GNb-RING affects substrate recognition or intrinsic activity and our expectation is that if we generate a GNb-RING construct in which all lysine residues are changed to arginine it should have WT activity in its ability to degrade substrate but should be resistant to autoubiquitination and thus have an extended half-life allowing it to maintain target protein at low levels for a long time.

### REFERENCES

Bondeson, D.P., Mares, A., Smith, I.E., Ko, E., Campos, S., Miah, A.H., Mulholland, K.E., Routly, N., Buckley, D.L., Gustafson, J.L., et al. (2015). Catalytic in vivo protein knockdown by small-molecule PROTACs. Nature chemical biology 11, 611-617.
Branigan, E., Plechanovova, A., Jaffray, E.G., Naismith, J.H., and Hay, R.T. (2015). Structural basis for the RING-catalyzed synthesis of K63-linked ubiquitin chains. Nature structural & molecular biology 22, 597-602.
Chirichella, M., Lisi, S., Fantini, M., Goracci, M., Calvello, M., Brandi, R., Arisi, I., D'Onofrio, M., Di Primio, C., and Cattaneo, A. (2017). Post-translational selective intracellular silencing of acetylated proteins with de novo selected intrabodies. Nature methods 14, 279-282.
Cox, J., and Mann, M. (2008). MaxQuant enables high peptide identification rates, individualized p.p.b.-range mass accuracies and proteome-wide protein quantification. Nature biotechnology 26, 1367-1372.
Dou, H., Buetow, L., Sibbet, G.J., Cameron, K., and Huang, D.T. (2012). BIRC7-E2 ubiquitin conjugate structure reveals the mechanism of ubiquitin transfer by a RING dimer. Nature structural & molecular biology 19, 876-883.
Doudna, J.A., and Charpentier, E. (2014). Genome editing. The new frontier of genome engineering with CRISPR-Cas9. Science 346, 1258096.
Elbashir, S.M., Harborth, J., Lendeckel, W., Yalcin, A., Weber, K., and Tuschl, T. (2001). Duplexes of 21-nucleotide RNAs mediate RNA interference in cultured mammalian cells. Nature 411, 494-498.
Hershko, A., and Ciechanover, A. (1998). The ubiquitin system. Annual review of biochemistry 67, 425-479.
Hobbs, S., Jitrapakdee, S., and Wallace, J.C. (1998). Development of a bicistronic vector driven by the human polypeptide chain elongation factor 1alpha promoter for creation of stable mammalian cell lines that express very high levels of recombinant proteins. Biochemical and biophysical research communications 252, 368-372.
Holland, A.J., Fachinetti, D., Han, J.S., and Cleveland, D.W. (2012). Inducible, reversible system for the rapid and complete degradation of proteins in mammalian cells. Proceedings of the National Academy of Sciences of the United States of America 109, E3350-3357.
Izant, J.G., Weatherbee, J.A., and McIntosh, J.R. (1983). A microtubule-associated protein antigen unique to mitotic spindle microtubules in PtK1 cells. The Journal of cell biology 96, 424-434.
Kirchhofer, A., Helma, J., Schmidthals, K., Frauer, C., Cui, S., Karcher, A., Pellis, M., Muyldermans, S., Casas-Delucchi, C.S., Cardoso, M.C., et al. (2010). Modulation of protein properties in living cells using nanobodies. Nature structural & molecular biology 17, 133-138.
Maghames, C.M., Lobato-Gil, S., Perrin, A., Trauchessec, H., Rodriguez, M.S., Urbach, S., Marin, P., and Xirodimas, D.P. (2018). NEDDylation promotes nuclear protein aggregation and protects the Ubiquitin Proteasome System upon proteotoxic stress. Nature communications 9, 4376.
Mann, M. (2006). Functional and quantitative proteomics using SILAC. Nature reviews. Molecular cell biology 7, 952-958.
Mendoza, H.M., Shen, L.N., Botting, C., Lewis, A., Chen, J., Ink, B., and Hay, R.T. (2003). NEDP1, a highly conserved cysteine protease that deNEDDylates Cullins. The Journal of biological chemistry 278, 25637-25643.
Nishimura, K., Fukagawa, T., Takisawa, H., Kakimoto, T., and Kanemaki, M. (2009). An auxin-based degron system for the rapid depletion of proteins in nonplant cells. Nature methods 6, 917-922.
Ong, S.E., and Mann, M. (2007). Stable isotope labeling by amino acids in cell culture for quantitative proteomics. Methods in molecular biology 359, 37-52.
Pardon, E., Laeremans, T., Triest, S., Rasmussen, S.G., Wohlkonig, A., Ruf, A., Muyldermans, S., Hol, W.G., Kobilka, B.K., and Steyaert, J. (2014). A general protocol for the generation of Nanobodies for structural biology. Nature protocols 9, 674-693.
Peterson, A.C., Russell, J.D., Bailey, D.J., Westphall, M.S., and Coon, J.J. (2012). Parallel reaction monitoring for high resolution and high mass accuracy quantitative, targeted proteomics. Molecular & cellular proteomics: MCP 11, 1475-1488.
Plechanovova, A., Jaffray, E.G., McMahon, S.A., Johnson, K.A., Navratilova, I., Naismith, J.H., and Hay, R.T. (2011). Mechanism of ubiquitylation by dimeric RING ligase RNF4. Nature structural & molecular biology 18, 1052-1059.
Plechanovova, A., Jaffray, E.G., Tatham, M.H., Naismith, J.H., and Hay, R.T. (2012). Structure of a RING E3 ligase and ubiquitin-loaded E2 primed for catalysis. Nature 489, 115-120.
Pleiner, T., Bates, M., Trakhanov, S., Lee, C.T., Schliep, J.E., Chug, H., Bohning, M., Stark, H., Urlaub, H., and Gorlich, D. (2015). Nanobodies: site-specific labeling for super-resolution imaging, rapid epitope-mapping and native protein complex isolation. eLife 4, e11349.
Prescott, A.R., Dowrick, P.G., and Warn, R.M. (1992). Stable and slow-turning-over microtubules characterize the processes of motile epithelial cells treated with scatter factor. Journal of cell science 102 (Pt 1), 103-112.
Prole, D.L., and Taylor, C.W. (2019). A genetically encoded toolkit of functionalized nanobodies against fluorescent proteins for visualizing and manipulating intracellular signalling. BMC biology 17, 41.
Pruneda, J.N., Littlefield, P.J., Soss, S.E., Nordquist, K.A., Chazin, W.J., Brzovic, P.S., and Klevit, R.E. (2012). Structure of an E3:E2˜Ub complex reveals an allosteric mechanism shared among RING/U-box ligases. Molecular cell 47, 933-942.
Quevedo, C.E., Cruz-Migoni, A., Bery, N., Miller, A., Tanaka, T., Petch, D., Bataille, C.J.R., Lee, L.Y.W., Fallon, P.S., Tulmin, H., et al. (2018). Small molecule inhibitors of RAS-effector protein interactions derived using an intracellular antibody fragment. Nature communications 9, 3169.
Rojas-Fernandez, A., Plechanovova, A., Hattersley, N., Jaffray, E., Tatham, M.H., and Hay, R.T. (2014). SUMO chain-induced dimerization activates RNF4. Molecular cell 53, 880-892.
Runcie, A.C., Chan, K.H., Zengerle, M., and Ciulli, A. (2016). Chemical genetics approaches for selective intervention in epigenetics. Current opinion in chemical biology 33, 186-194.
Sakamoto, K.M., Kim, K.B., Kumagai, A., Mercurio, F., Crews, C.M., and Deshaies, R.J. (2001). Protacs: chimeric molecules that target proteins to the Skp1-Cullin-F box complex for ubiquitination and degradation. Proceedings of the National Academy of Sciences of the United States of America 98, 8554-8559.
Shen, L.N., Liu, H., Dong, C., Xirodimas, D., Naismith, J.H., and Hay, R.T. (2005). Structural basis of NEDD8 ubiquitin discrimination by the deNEDDylating enzyme NEDP1. The EMBO journal 24, 1341-1351.
Shen, T.H., Lin, H.K., Scaglioni, P.P., Yung, T.M., and Pandolfi, P.P. (2006). The mechanisms of PML-nuclear body formation. Molecular cell 24, 331-339.
Shevchenko, A., Tomas, H., Havlis, J., Olsen, J.V., and Mann, M. (2006). In-gel digestion for mass spectrometric characterization of proteins and proteomes. Nature protocols 1, 2856-2860.
Sun, H., Leverson, J.D., and Hunter, T. (2007). Conserved function of RNF4 family proteins in eukaryotes: targeting a ubiquitin ligase to SUMOylated proteins. The EMBO journal 26, 4102-4112.
Toyama, B.H., Savas, J.N., Park, S.K., Harris, M.S., Ingolia, N.T., Yates, J.R., 3rd, and Hetzer, M.W. (2013). Identification of long-lived proteins reveals exceptional stability of essential cellular structures. Cell 154, 971-982.
Tyanova, S., Temu, T., Sinitcyn, P., Carlson, A., Hein, M.Y., Geiger, T., Mann, M., and Cox, J. (2016). The Perseus computational platform for comprehensive analysis of (prote)omics data. Nature methods 13, 731-740.
Wilton, E.E., Opyr, M.P., Kailasam, S., Kothe, R.F., and Wieden, H.J. (2018). sdAb-DB: The Single Domain Antibody Database. ACS synthetic biology 7, 2480-2484.

1-25. (canceled)

26. A molecule comprising an E3 ligase component and a target protein binding moiety.

27. The molecule of claim 26, wherein the E3 ligase component recruits the ubiquitin loaded E2 conjugating enzyme.

28. The molecule of claim 26, wherein the target protein binding moiety binds a protein to be degraded.

29. The molecule of claim 26, wherein the E3 ligase component comprises a U-box sequence.

30. The molecule of claim 26, wherein the E3 ligase component is the RING domain of a SUMO-targeted ubiquitin ligase.

31. The molecule of claim 26, wherein the E3 ligase component is the RING domain of ubiquitin E3 ligase RNF4.

32. The molecule of claim 26, wherein the E3 ligase component comprises a sequence of any one of SEQ ID NOS: 1-29, or a RING domain or U-box fragment thereof.

33. The molecule of claim 32, wherein a RING domain or U-box fragment recruits the ubiquitin loaded E2 conjugating enzyme.

34. The molecule of claim 26, wherein the target protein binding moiety is a nanobody.

35. The molecule of claim 34, wherein the nanobody has specificity or affinity for, or binds to, a protein to be degraded.

36. The molecule of claim 35, wherein the protein to be degraded is an intracellular protein, optionally, wherein the protein is degraded by the ubiquitin-proteasome system.

37. The molecule of claim 34, wherein the nanobody is a bispecific nanobody.

38. The molecule of claim 37, wherein the bi-specific nanobody has specificity or affinity for, or binds to, an extracellular protein and an intracellular protein.

39. The molecule of claim 26, wherein the molecule is a fusion construct.

40. The molecule of claim 26, wherein the molecule further comprises a nuclear localisation signal.

41. A method of treating a disease, cancer, or a neurodegenerative disorder, said method comprising administering a subject in need thereof, a therapeutically effective amount of a molecule according to claim 26.

42. A method of degrading a protein, said method comprising contacting a protein to be degraded with a molecule of claim 26.

43. The method of claim 42, wherein the protein is a cellular protein, and said method comprises contacting a cell expressing the protein to be degraded with a molecule of claim 26.

44. A cell comprising a molecule of claim 26.

45. A nucleic acid or vector encoding a molecule of claim 26.
